(19) United States
(12) Patent Application Publication
(10) Pub. No.: US 2026/0014039 A1
(43) Pub. Date: Jan. 15, 2026

(54) PATIENT SUPPORT APPARATUS COMMUNICATION SYSTEM

(71) Applicant: Stryker Corporation, Portage, MI (US)

(72) Inventors: Madhu Sandeep Thota, Portage, MI (US); Anish Paul, Portage, MI (US); Krishna Sandeep Bhimavarapu, Kalamazoo, MI (US); Celso Henrique Farnese Pires Pereira, Portage, MI (US); Madhu Thomas, London, Ontario (CA); Jerald A. Trepanier, Augusta, MI (US); Kirby M. Neihouser, Portage, MI (US); William Dwight Childs, Plainwell, MI (US); Kyle M. Thomas, Portage, MI (US); Matthew S. Baker, Portage, MI (US)

(21) Appl. No.: 19/132,927

(22) PCT Filed: Nov. 27, 2023
(86) PCT No.: PCT/US2023/081094

§ 371 Date: May 27, 2025

Related U.S. Application Data
(60) Provisional application No. 63/428,077, filed on Nov. 27, 2022.

(51) Int. Cl. A61G 7/018; A61G 7/05; G16H 40/67

(52) U.S. Cl. CPC A61G 7/018; A61G 7/0524; G16H 40/67; A61G 2203/12

(57) ABSTRACT

A patient support apparatus includes a patient support surface, at least one UWB transceiver, a network transceiver, and a controller. The controller is adapted to perform the following: use UWB communication between the UWB transceiver and a user device to determine if the user device is positioned within a first distance of the patient support apparatus, to forward data from the user device to a server if the user device is positioned within the first distance of the patient support apparatus, and to not do so if the user device is positioned outside of the first distance of the patient support apparatus. The user device may include a microphone and the data may include audio signals from a patient's voice. The UWB transceiver may require detecting the presence of a locator unit prior to forwarding the data from the user device to the server.

### BACKGROUND

The present disclosure relates to patient support apparatuses, such as beds, cots, stretchers, recliners, or the like. More specifically, the present disclosure relates to patient support apparatuses that communicate with one or more user device, such as phones, tablet computers, pendants, and/or other devices that are separate from the patient support apparatus itself.

### SUMMARY

According to the various aspects described herein, the present disclosure is directed to a patient support apparatus system that monitors the positions of user devices. In some cases, the user devices are used patients, by caregivers, and/or by both patient and caregivers. The user devices may comprise cell phones, tablet computers, pendants, or other portable electronic devices. The system allows data from the user devices to be automatically routed to the correct recipients without requiring that manual steps be undertaking matching a particular user device with one or more particular recipients. Instead, the system enables the automatic routing of data from the user devices to one or more intended destinations, even when the user device is first added to the system and/or even when the user device is used with different patients, different patient support apparatuses, and/or with different caregivers. In some aspects, the data that is automatically routed includes phone calls and/or texts, thereby allowing a patient, for example, to use his or her cell phone to call or text the nurse responsible for their care without having to know the nurse's phone number, name, or other identifying information. These and other aspects of the present disclosure will be apparent to one of ordinary skill in the art in light of the following written description and the accompanying drawings.

According to a first aspect of the present disclosure, a patient support apparatus is provided that includes a support surface adapted to support a patient, an ultra-wideband (UWB) transceiver adapted to wirelessly communicate with a user device, a network transceiver adapted to communicate with a healthcare facility computer network, and a controller. The controller is adapted to use UWB communication between the UWB transceiver and the user device to determine if the user device is positioned within a first distance of the patient support apparatus. The controller is further adapted to forward data from the user device to a server on the healthcare facility network if the user device is positioned within the first distance of the patient support apparatus, and to not forward the data from the user device to the server if the user device is positioned outside of the first distance of the patient support apparatus.

According to another aspect of the present disclosure, the controller is adapted to use UWB communication between the UWB transceiver and a fixed locator unit to determine if the fixed locator unit is positioned within a second distance of the patient support apparatus.

In some aspects, the controller is further adapted to forward the data from the user device to the server on the healthcare facility network only if the user device is positioned within the first distance of the patient support apparatus and the patient support apparatus is positioned within the second distance of the fixed locator unit, and to not forward the data from the user device to the server if the user device is positioned outside of the first distance of the patient support apparatus or if the patient support apparatus is positioned outside of the second distance of the fixed locator unit.

The data, in some aspects, includes audio signals generated from a microphone of the user device.

The user device, in some aspects, is one of a patient's cell phone, a patient's tablet computer, or a patient's wireless bed pendant.

In some aspects, the user device is a wireless bed pendant including a control for transmitting a wireless motion command to the patient support apparatus, and the controller is adapted to implement the motion command only if the wireless bed pendant transmits the wireless motion command to the patient support apparatus using at least two different wireless communication protocols.

In some aspects, the at least two different wireless communication protocols include Bluetooth and UWB communications.

The controller, in some aspects, is further adapted to receive a locator unit ID from the fixed locator unit when the fixed locator unit is positioned within the second distance of the patient support apparatus.

In some aspects, the controller is adapted to forward the data to the server by forwarding the data to the fixed locator unit.

In other aspects, the controller is adapted to forward the data to the server by forwarding the data to the server using the network transceiver.

The controller, in some aspects, is further adapted to forward the locator unit ID to the server.

In some aspects, the patient support apparatus further includes a first Bluetooth transceiver adapted to communicate with a second Bluetooth transceiver integrated into the user device, and wherein the audio signals are delivered to the patient support apparatus via the first and second Bluetooth transceivers.

The controller, in some aspects, is further adapted to receive pairing data from the user device via the UWB transceiver and to use the pairing data to automatically pair the first and second Bluetooth transceivers together if the user device is positioned within the first distance of the patient support apparatus.

The network transceiver, in some aspects, is a WiFi transceiver.

According to another aspect of the present disclosure, a patient support apparatus system is provided that includes a patient support apparatus and a server. The patient support apparatus includes a support surface adapted to support a patient, an ultra-wideband (UWB) transceiver adapted to wirelessly communicate with a user device, a network transceiver adapted to communicate with the server, and a controller. The controller is adapted to use UWB communication between the UWB transceiver and the user device to determine if the user device is positioned within a first distance of the patient support apparatus. The controller is further adapted to forward a location ID and data from the user device to the server if the user device is positioned within the first distance of the patient support apparatus, and to not forward the location ID and the data from the user device to the server if the user device is positioned outside of the first distance of the patient support apparatus. The server is adapted use the location ID to determine a destination to which to route the data from the user device.

According to other aspects of the disclosure, the server is further adapted to communicate with a second server to receive a caregiver ID of a caregiver associated with the patient, and to use the caregiver ID to determine the destination to which to route the data.

In some aspects, the server is further adapted to communicate with a third server to receive a badge ID of a badge associated with the caregiver, and to route the data to the badge.

The controller, in some aspects, is further adapted to use UWB communication between the UWB transceiver and a fixed locator unit to determine if the fixed locator unit is positioned within a second distance of the patient support apparatus.

In some aspects, the controller is further adapted to forward the data from the user device to the server on the healthcare facility network only if the user device is positioned within the first distance of the patient support apparatus and the patient support apparatus is positioned within the second distance of the fixed locator unit, and to not forward the data from the user device to the server if the user device is positioned outside of the first distance of the patient support apparatus or if the patient support apparatus is positioned outside of the second distance of the fixed locator unit.

In some aspects, the data includes audio signals generated from a microphone of the user device.

In some aspects, the user device is one of a patient's cell phone, a patient's tablet computer, or a patient's wireless bed pendant.

In some aspects, the user device is a wireless bed pendant including a control for transmitting a wireless motion command to the patient support apparatus, and the controller is adapted to implement the motion command only if the wireless bed pendant transmits the wireless motion command to the patient support apparatus using at least two different wireless communication protocols.

The at least two different wireless communication protocols, in some aspects, include Bluetooth and UWB communications.

The controller, in some aspects, is further adapted to receive the location ID from the fixed locator unit when the fixed locator unit is positioned within the second distance of the patient support apparatus.

The controller, in some aspects, is adapted to forward the data to the server by forwarding the data to the fixed locator unit.

The controller, in some aspects, is adapted to forward the data to the server by forwarding the data to the server using the network transceiver.

The patient support apparatus, in some aspects, further includes a first Bluetooth transceiver adapted to communicate with a second Bluetooth transceiver integrated into the user device, and the audio signals are delivered to the patient support apparatus via the first and second Bluetooth transceivers.

In some aspects, the controller is further adapted to receive pairing data from the user device via the UWB transceiver and to use the pairing data to automatically pair the first and second Bluetooth transceivers together if the user device is positioned within the first distance of the patient support apparatus.

The network transceiver, in some aspects, is a WiFi transceiver.

The server, in some aspects, is adapted to use the location ID to determine multiple destinations to which to route the data from the user device.

In some aspects, a first one of the multiple destinations is a display device mounted at a fixed location within the healthcare facility.

In some aspects, a second one of the multiple destination is a mobile device adapted to be carried by a caregiver assigned to the patient.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of a patient support apparatus system of the present disclosure showing the patient support apparatus, a user device, a caregiver with a badge, and a linked locator unit that is used for automatically detecting the location of a patient support apparatus and/or other devices;

FIG. 5 is a block diagram of the patient support apparatus system of FIG. 4;

FIG. 6 is a plan view of a first screen displayable on a mobile user device;

FIG. 7 is a plan view of a second screen displayable on the mobile user device;

FIG. 8 is a plan view of a third screen displayable on the mobile user device;

FIG. 9 is a plan view of a fourth screen displayable on the mobile user device; and

FIG. 10 is a communication diagram of several components of the patient support apparatus system of the present disclosure.

### DETAILED DESCRIPTION OF THE EMBODIMENTS

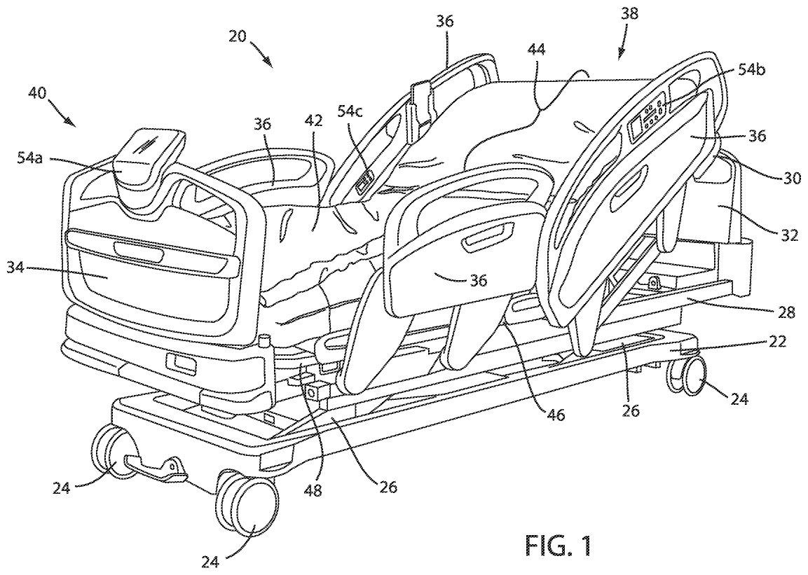

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width and/or length of patient support deck 30, thereby allowing patient support apparatus 20 to accommodate patients of varying sizes. When so modified, the width of deck 30 may be adjusted sideways and/or lengthwise in increments or otherwise.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,59 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

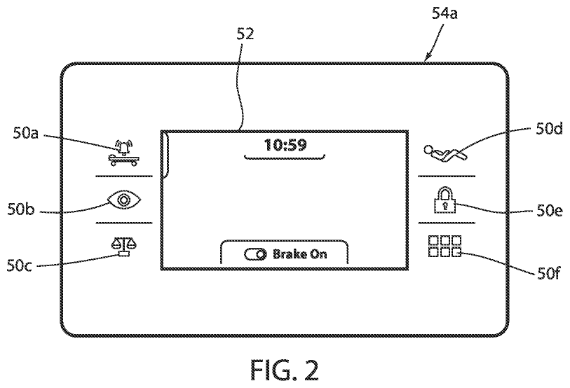

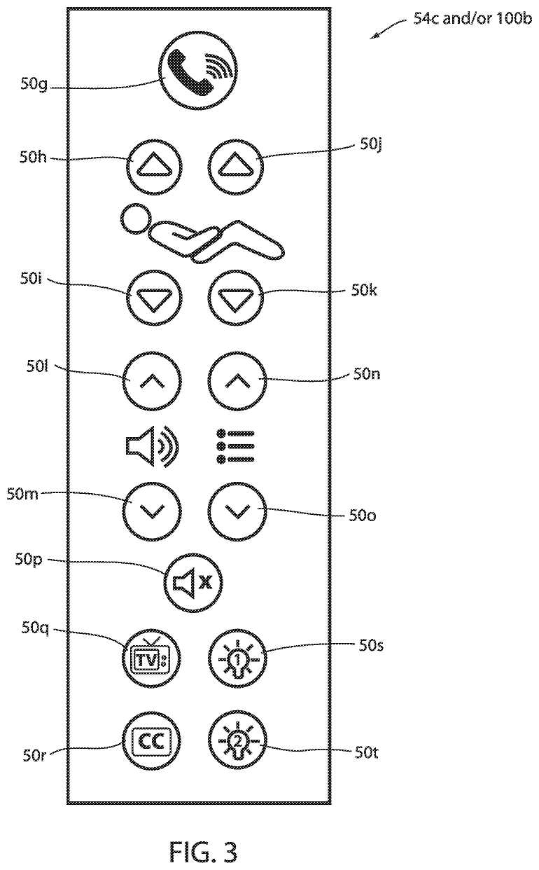

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

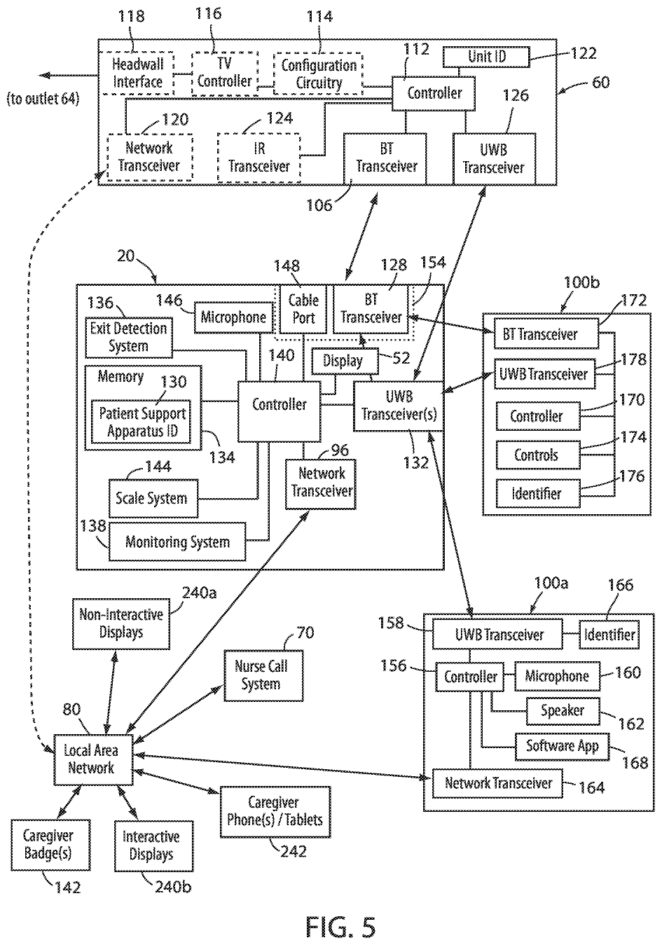

Among other functions, controls 50 of control panel 54a allow a user to control one or more of the following: change a height of support deck 30; raise or lower head section 44; activate and deactivate a brake for wheels 24; arm and disarm an exit detection system 136 and/or an onboard monitoring system 138 (FIG. 5); change various settings on patient support apparatus 20; view the current location of the patient support apparatus 20 as determined by the location detection system discussed herein; view what devices, such as, but not limited to, medical devices, exercise devices, nurse call devices, caregiver badges, etc. that the patient support apparatus 20 has associated itself with; and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system 136 (FIG. 5). The exit detection system 136 is as adapted to issue an alert when a patient exits from patient support apparatus 20. Exit detection system 136 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 54 displays a monitoring control screen that includes a plurality of control icons that, when touched, control the onboard monitoring system 138 (FIG. 5) built into patient support apparatus 20. The onboard monitoring system 138 alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Stated alternatively, monitoring system 138, when armed, monitors a plurality of conditions of patient support apparatus 20 (such as, but not limited to, any one or more of the following: brake status, siderail position, litter frame height, exit detection system 136, A/C cord status, nurse call cable status, etc.) and issues an alert if any one of those conditions are in an undesired state. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 54a displays a scale control screen that includes a plurality of control icons that, when touched, control a scale system 144 (FIG. 5) of patient support apparatus 20. Such a scale system 144 may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors and/or other components that are utilized by the exit detection system 136, or it may utilize one or more different sensors and/or other components. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such motion lockout functions typically include the ability for a caregiver to use control panel 54a to lock out one or more of the motion controls 50 of the patient control panels 54c such that the patient is not able to use those controls 50 on control panels 54c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

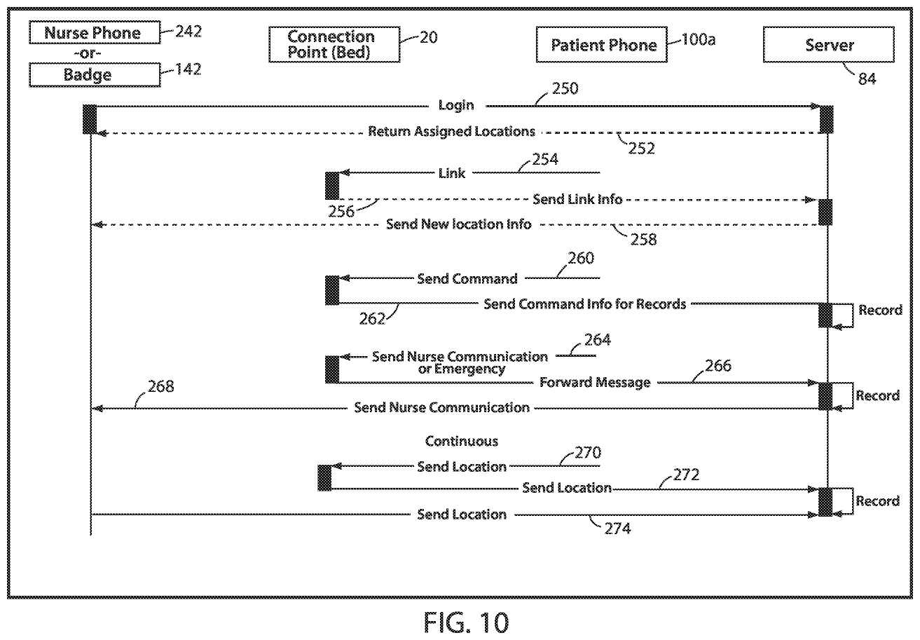

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, displaying information about one or more devices that are currently associated with patient support apparatus 20, diagnostic and/or service information for patient support apparatus 20, mattress control and/or status information, configuration settings, location information, and other settings and/or information. Examples of menu screens are shown in FIGS. 10 and 11, as well as the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to see which devices, if any, are currently associated with patient support apparatus 20. As will be discussed in greater detail below, patient support apparatus 20 includes an onboard locating system that is adapted to automatically determine the relative position of one or more devices with respect to patient support apparatus 20 and, in some instances, automatically associate and/or disassociate those devices with and/or from patient support apparatus 20 (and/or the patient assigned to patient support apparatus 20) depending upon the proximity of the device to patient support apparatus 20. Further details of this locating system are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel that may either be integrated into patient support apparatus 20 (e.g. control panel 54c), or that may be integrated into a stand-alone pendant that is communicatively coupled to patient support apparatus 20, either by a cord or wirelessly. As will be discussed in more detail below, when the patient control panel of FIG. 3 is integrated into a wireless pendant, it may be considered to be one type of user device 100 that patient support apparatus 20 is configured to automatically pair with if it is positioned within range, as will also be discussed in greater detail below. Regardless of whether it is part of an integrated control panel 54c or a user device 100, the patient control panel of FIG. 3 includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30. The knee section may refer to the joint that couples thigh section 46 to foot section 48.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20. In other embodiments, instead of being pure toggle switches, controls 50s and/or 50t may gradually increase or decrease the brightness of the reading or room light as long they are held down, and if subsequently turned off and on, they may reset the brightness level to an initial low (or high) intensity level (that the user can adjust by continuing to press down on the corresponding control 50s or 50t.

It will be understood that not only the number of controls 50 on the control panel of FIG. 3, but also the functions of these controls 50, the layout of these controls 50, and/or other aspects of these controls 50 may be modified from what is shown in FIG. 3.

In those embodiments where the control panel of FIG. 3 is integrated into a wireless pendant user device 100, patient support apparatus 20 may be configured to only respond to motion commands from the wireless pendant user device 100 if patient support apparatus 20 receives the motion commands via two separate communication protocols. For example, in such embodiments, patient support apparatus 20 may be configured to respond to motion commands from the wireless pendant user device 100 if it receives the same motion command sent over both a Bluetooth communication channel and another communication channel (e.g. ultra-wideband). If patient support apparatus 20 receives a motion command over only a single communication channel, it is configured to ignore the motion command. This feature is provided as a safety feature such that movement of components of patient support apparatus 20 is only able to be carried out via wirelessly when the motion commands are duplicated across two different wireless communication protocols. In some embodiments, the motion commands are continuous signals that are sent over both communication channels and patient support apparatus 20 only responds to these motion commands while it is receiving both of these continuous signals. The term “motion commands,” as used herein, refers to controls 50 that implement movement of one or more components of patient support apparatus 20, such as, but not limited to, controls 50h, 50i, 50j, and 50k.

As will be discussed in greater detail below, in addition to requiring the receipt of duplicate motion commands before implementing movement of one or more components of patient support apparatus 20, patient support apparatus 20 is also configured to only respond to a wireless pendant user device 100 if the wireless pendant user device 100 has been paired with, or associated with, that particular patient support apparatus 20. The terms “paired” or “associated,” and/or their variants, are generally used interchangeably with each other herein. The manner in which patient support apparatus 20 associates, or pairs, with one or more user devices 100 is discussed in greater detail below.

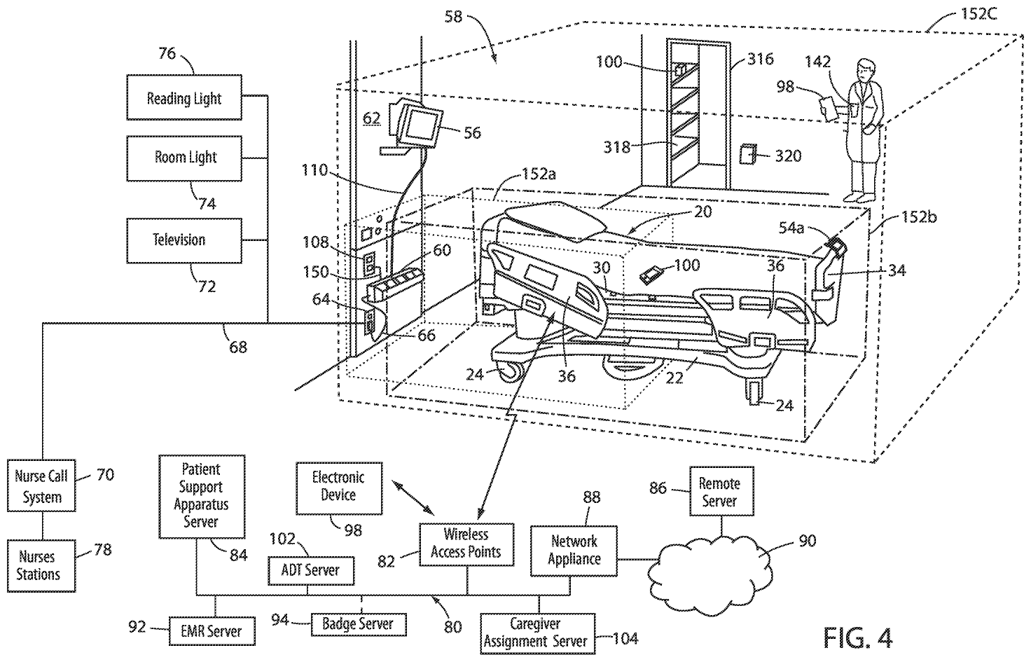

FIG. 4 illustrates patient support apparatus 20 positioned within a room 58 of a healthcare facility. FIG. 4 also illustrates additional items that may be present in a healthcare facility and which patient support apparatus 20 is configured to communicate with, including, but not limited to, a locator unit 60, a conventional local area network 80 of the healthcare facility, a caregiver badge 142, and one or more user devices 100 used during the care of a patient. Locator units 60 are positioned at known and fixed locations within the healthcare facility in which patient support apparatus 20 is positioned. Locator units 60 function as fixed locators. That is, locator units 60 communicate with patient support apparatuses 20 and share information with them that allows the location of the patient support apparatuses 20 to be determined, as well as the location of any user devices 100 that are associated with patient support apparatus 20.

In some embodiments, patient support apparatus 20 is configured to be able to communicate with at least two different types of locator units 60: linked locator units and unlinked locator units. One example of a linked locator unit 60 is shown in FIG. 4. Examples of unlinked locator units 60 are shown (and referred to as unlinked locator units 60b) in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES, the complete disclosure of which is incorporated herein by reference. Other examples of unlinked locator units 60 are shown (and referred to as unlinked locator units 60a) in commonly assigned U.S. patent application Ser. No. 63/356,061 filed Jun. 28, 2022, by inventors Krishna Bhimavarapu et al. and entitled BADGE AND PATIENT SUPPORT APPARATUS COMMUNICATION SYSTEM, the complete disclosure of which is incorporated herein by reference. Patient support apparatus 20 is configured to communicate with the unlinked locator units described in either of the aforementioned '279 or '061 applications and/or to perform any one or more of the functions described therein that utilize such unlinked locator units and/or information provided by such unlinked locator units. Unless explicitly stated otherwise, all references herein to “locator units 60” without the term “linked” or “unlinked” in the reference will refer to both linked and unlinked locator units 60.

Linked locator units 60 are adapted to be communicatively linked to a conventional communication outlet 64 and are adapted to provide location information to patient support apparatus 20. Linked locator units 60 are also adapted to serve as a communication conduit for routing wireless communications between patient support apparatus 20 and one or more devices and/or systems that are communicatively coupled to communication outlet 64 (e.g. room devices 72, 74, 76, and/or nurse call system 70, FIG. 4). In general, linked locator units 60 are typically positioned in patient rooms of the healthcare facility where one or more communication outlets 64 are typically present.

As shown in FIG. 4, linked locator unit 60 is adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to linked locator unit 60 (shown in FIG. 4). In many healthcare facilities, communication outlet 64 includes a 37-pin connector, although other types of connectors are often found in certain healthcare facilities. As will be discussed in greater detail below, linked locator unit 60 and nurse call cable 66 allow patient support apparatus 20 to communicate with a nurse call system 70, and one or more room devices positioned within room 58.

Communication outlet 64 is electrically coupled to one or more cables, wires, or other type of conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 76 and/or nurse call system 70. Still other communicative arrangements for coupling communication outlet 64 to nurse call system 70 and/or one or more room devices 72, 74, 76 are possible.

Nurse call cable 66 (FIG. 4) enables linked locator unit 60 to communicate with nurse call system 70 and/or room devices 72, 74, 76. Because patient support apparatus 20 is able to wirelessly communicate with linked locator unit 60, patient support apparatus 20 is thereby able to communicate with nurse call system 70 and room devices 72, 74, 76. A patient supported on patient support apparatus 20 who activates a nurse call control (e.g. 50g; see FIG. 3) on patient support apparatus 20 causes a signal to be wirelessly sent from patient support apparatus 20 to linked locator unit 60, which in turn conveys the signal via nurse call cable 66 to the nurse call system 70, which forwards the signal to one or more remotely located nurses (e.g. nurses at one or more nurse's stations 78). If the patient activates one or more room device controls (e.g. controls 50l-t; see FIG. 3), one or more wireless signals are conveyed to linked locator unit 60, which in turn sends appropriate signals via nurse call cable 66 to communication outlet 64 and the room device 72, 74, 76 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 96 (FIG. 5) that communicates wirelessly with local area network 80. Network transceiver 96 is, in at least some embodiments, a WiFi transceiver (e.g. IEEE 802.11) that wirelessly communicates with one or more conventional wireless access points 82 of local area network 80. In other embodiments, network transceiver 96 may be a wireless transceiver that uses conventional 5G technology to communicate with network 80, one or more servers hosted thereon, and/or other devices. In some embodiments, network transceiver 96 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 96 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 96 for communicating with network 80 via a wired connection and a wireless transceiver 96 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive data from the patient support apparatuses 20 positioned within the healthcare facility and distribute this data to caregivers, other servers, and/or other software applications. As will be discussed in greater detail below, server 84 may also be configured to receive data from one or more user devices 100 and/or badges 142 that are positioned within one or more volumes of space defined around patient support apparatus 20 and/or within a volume of space defined around locator units 60. Such data is then routed by patient support apparatus server 84 to one or more desired destinations. In some embodiments, discussed in more detail below, the data that is received from one or more user devices 100 may include audio signals from a microphone in the user device, and patient support apparatus server 84 may forward those audio signals to an appropriate caregiver device (e.g. a smart phone, badge 142, or other device with audio-playback capabilities).

In some embodiments, the data from the user device(s) 100 is forwarded from the device(s) 100 to patient support apparatus 20, and from patient support apparatus 20 to server 84 via network transceiver 96 and one or more access points 82. In other embodiments, such as where a locator unit 60 includes its own network transceiver, the data from a user device 100 may be forwarded to the locator unit 60 and the locator unit 60 may then forward the data to server 84 via its network transceiver's communication with one or more of the access point 82. In such embodiments, the user device 100 may forward its data directly to the locator unit 60 without sending it to patient support apparatus 20, or it may send its data to patient support apparatus 20 first and patient support apparatus 20 may then forward the data to the locator unit 60. Other manners of routing data from the user device 100 to server 84 may be used, and in some embodiments, the user device 100 may send some types of data to server 84 along a first route and send other types of data to server 84 along a second and different route.

In some embodiments, patient support apparatus server 84 is configured to communicate at least some of the patient support apparatus data and/or device data received from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). Patient support apparatus 20 is provided with the URL and/or other information necessary to communicate with remote server 86 via the Internet connection between network 80 and remote server 86. In some such embodiments, network appliance 88 is a router configured to support such direct connections. Still other types of direct-to-cloud connections may be utilized with one or more of patient support apparatuses 20. When patient support apparatus 20 is configured to directly communicate with remote server 86, patient support apparatus server 84 may be omitted and any one or more of the functions of patient support apparatus server 84 described herein may be performed by remote server 86.

Patient support apparatus server 84 is also configured to determine the location of each patient support apparatus 20 (and/or its associated devices), or receive the location of each patient support apparatus 20 (and/or its associated devices) from the patient support apparatuses 20. In some embodiments, patient support apparatus server 84 determines the room number and/or bay area of each patient support apparatus 20 and its associated devices that are positioned within a room 58, as well as the location of patient support apparatuses 20 and their associated devices that are positioned outside of a room 58, such as those that may be positioned in a hallway, a maintenance area, or some other area. In general, patient support apparatus server 84 may be configured to determine the position of any patient support apparatus 20 that is positioned within communication range of one or more locator units 60, as well as the location of any associated devices that are positioned within one or more volumes of space defined around the patient support apparatus 20, as will be discussed in greater detail below.

Patient support apparatus server 84 (FIG. 4) is adapted to communicate with a plurality of other servers, such as a conventional EMR server 92, a conventional badge server 94, a conventional Admission, Discharge, and Transfer (ADT) server 102, and/or a conventional caregiver assignment server 104. Alternatively, patient support apparatus server 84 may be combined, either partially or wholly, with any one or more of these other server. EMR server 92 stores individual patient records. Such patient records identify a patient by name and include medical information associated with that patient. Such medical information may include all of the medical information generated from the patient's current stay in the healthcare facility as well as medical information from previous visits. It will be understood that the term “EMR server,” as used herein, also includes Electronic Health Records servers, or EHR servers for short, and that the present disclosure does not distinguish between electronic medical records and electronic health records.

Caregiver assignment server 104 (FIG. 4) stores data that matches specific caregivers to specific rooms and/or bays within the healthcare facility. Caregiver assignment server 104 stores information regarding shift changes, personnel, and the general assignments of caregivers who are employed by the healthcare facility. In some caregiver assignment servers 104, caregivers are assigned to specific patients, rather than to specific rooms, in which case server 104 may correlate caregivers to individual patients rather than rooms and/or bays. Still further, some conventional nurse call systems may be configured to carry out the functions of caregiver assignment server 104, in which case caregiver assignment server 104 may be replaced by and/or supplemented with a nurse call server.

ADT server 102 stores patient information, including the identity of patients and the corresponding rooms 58 and/or bays within rooms to which the patients are assigned. That is, ADT server 102 matches specific patients to specific rooms and/or bays within the healthcare facility. The patient's names are entered into the ADT server 102 by one or more healthcare facility staff whenever a patient checks into the healthcare facility and the patient is assigned to a particular room within the healthcare facility. If and/or when a patient is transferred to a different room and/or discharged from the healthcare facility, the staff of the healthcare facility update ADT server 102. ADT server 102 therefore maintains an up-to-date set of data that correlates patient names with their assigned rooms and/or bays. In some conventional electronic medical record systems, the functions of the ADT server 102 may be incorporated into the EMR system, and EMR server 92 may therefore, in some embodiments, carry out the functions of ADT server 102.

Badge server 94 (FIG. 4) is configured to manage communications between, and keep track of the locations of, and the specific caregivers assigned to, individual badges 142. Badges 142 are typically worn by healthcare workers, such as caregivers, service technicians, cleaning personnel, transportation assistants, etc. Badge server 94 maintains a set of data that correlates badge IDs with individual healthcare workers. Each badge 142 includes a unique ID that distinguishes that badge 142 from other badges 142. When a healthcare provider arrives at a healthcare facility, he or she typically grabs a badge 142 from a common collection of badges 142 and wears it for the duration of his or her work shift (and returns it to the general collection after his/her shift). For some badge servers 94, in order for the badge server 94 to know that a particular badge 142 is assigned to a particular healthcare worker, the worker manually associates the particular badge 142 they choose to wear that day with their name (or some other worker ID). This manual association may involve scanning the badge on a badge reader in communication with server 94, typing information (such as the badge ID and/or the worker's ID) into a computer coupled to server 94, entering information into badge 142 (if it is a badge that allows data entry), performing other actions, and/or a combination of these steps.

Badge server 94 may also be configured to monitor the location of badges 142 within a healthcare facility. Typically this location monitoring is performed through the monitoring of the wireless access points 82 throughout the healthcare facility. That is, badges 142 are often equipped to use WiFi, or other wireless communication protocols, that allow them to communicate with wireless access points 82. By monitoring which access points the badges 142 are currently connected to, using a map of the location of the access point 82 within the facility, and, in some cases, using the signal strengths between the badges 142 and one or more of the access points 82, the general position of the badges 142 within the healthcare facility can be determined by badge server 94. In some embodiments, badges 142 may include UWB transceivers that enable their location within the healthcare facility to be more precisely determined, and/or that enable the badges 142 to perform other functions. Examples of badges 142 that include UWB transceivers and that may be incorporated into the system of the present disclosure are described in greater detail in commonly assigned U.S. provisional patent application Ser. No. 63/356,061 filed Jun. 28, 2022, by inventors Krishna Bhimavarapu et al. and entitled BADGE AND PATIENT SUPPORT APPARATUS COMMUNICATION SYSTEM; in commonly assigned U.S. provisional patent application Ser. No. 63/356,065 filed Jun. 28, 2022, by inventors Jerald Trepanier et al. and entitled BADGE AND PATIENT SUPPORT APPARATUS COMMUNICATION SYSTEM; and in commonly assigned U.S. provisional patent application Ser. No. 63/356,238 filed Jun. 28, 2028, by inventors Madhu Sandeep Thota et al. and entitled BADGE AND PATIENT SUPPORT APPARATUS COMMUNICATION SYSTEM; the complete disclosures of all of which are incorporated herein by reference. Patient support apparatus 20, patient support apparatus server 84, badges 142, and/or locator units 60 of the present disclosure may be configured to carry out any of the functions of the patient support apparatuses, patient support apparatus server, badges, and locator units, respectively, disclosed in any of the aforementioned '061, '065, and/or '238 patent applications.

Badges 142 may be badges of the type sold or marketed by Stryker Corporation of Kalamazoo, Michigan, under the names Vocera Badge, Vocera Smartbadge, and/or Vocera Minibadge. Other types of badges may also, or alternatively, be used. Such badges 142 include the ability to transmit voice communications of healthcare workers to other badges 142 and/or other locations within a healthcare facility. Some of the badges may also include text messaging abilities, alarm notifications, and other functions. When integrated into the system described herein, such badges 142 may be modified to include one or more ultra-wideband transceivers and/or tags that communicate with ultra-wideband transceivers onboard patient support apparatus 20 and/or built into locator unit 60, as will be discussed in greater detail herein. That is, patient support apparatus 20 and/or locator units 60 may be configured to repetitively determine the location of any of the badges 142 that are positioned within range of its ultra-wideband transceivers and determine whether the badge 142 is positioned inside or outside of one or more volumes of space, as will also be discussed in greater detail below. Badges 142 that do not include UWB transceivers may also be incorporated into the communication system described herein

In some embodiments, patient support apparatus server 84 (FIG. 4) communicates with EMR server 92 in order to transmit patient data that is to be recorded in a patient's health record (e.g. vital sign readings from one or more vital sign sensors; weight readings taken from the scales built into patient support apparatuses 20; therapies provided to patients using a powered mattress 42 onboard patient support apparatuses 20; data from other devices that are determined to be associated with the patient assigned to patient support apparatus 20, etc.). In addition, server 84 communicates with EMR server 92, in some embodiments, in order to receive data from one or more of the devices that are being used with a particular patient.

It will be understood that the architecture and content of local area network 80 will vary from healthcare facility to healthcare facility, and that the example shown in FIG. 4 is merely one example of the type of network a healthcare facility may be employ. Typically, one or more additional servers will be hosted on network 80 and one or more of them may be adapted to communicate with patient support apparatus server 84. Local area network 80 will also typically allow one or more electronic devices 98 to access the local area network 80 and the servers hosted thereon via wireless access points 82. Such electronic devices 98 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84). Patient support apparatus server 84 is configured, in some embodiments, to share data with one or more electronic devices 98 that relates to patient support apparatus 20, that relates to one or more user devices 100 that become associated with patient support apparatus 20 (or the patient assigned thereto), that relates to one or more badges 142 that become associated with patient support apparatus 20, and/or that relates to one or more medical records of the patient stored in EMR server 92. As will be discussed in more detail below, electronic devices 98 include, but are not limited to, caregiver phones/tablets 242, displays 240, and caregiver badges 142.

Linked locator units 60 are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 (via a cable port 148; FIG. 5) and communications outlet 64. Linked locator units 60 are also adapted to transmit signals received from communications outlet 64 to patient support apparatus 20 via a BT transceiver 106 and/or a UWB transceiver 126 (FIG. 5). Thus, patient support apparatus 20 and linked locator unit 60 cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked locator unit 60 (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked locator units 60 are also adapted to forward signals received from communications outlet 64 to patient support apparatus 20. Linked locator units 60 are therefore adapted to provide bidirectional communication between patient support apparatus 20 and communications outlet 64. This bidirectional communication includes, but is not limited to, communicating command signals from any of controls 50 and/or from any of electronic devices 98 to corresponding room devices 72, 74, and/or 76 and communicating audio signals between a person supported on patient support apparatus 20 and a caregiver positioned remotely from patient support apparatus 20. The audio signals received by linked locator unit 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20.

Nurse call cable 66, in some embodiments, includes a conventional 37 pin connector on each end, one of which is adapted to be inserted into outlet 64 and the other one of which is adapted to be inserted into a linked locator unit 60 (or cable port 148 of patient support apparatus 20 if wired communication is desired). Such 37 pin connections are one of the most common types of connectors found on existing walls of medical facilities for making connections to the nurse call system 70 and room devices 72, 74, and 76. Linked locator unit 60 and nurse call cable 66 are therefore configured to mate with one of the most common type of communication outlets 64 used in medical facilities. Such 37 pin connectors, however, are not the only type of connectors, and it will be understood that linked locator units 60 can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Locator unit 60 (FIG. 4) also includes an electrical cord 150 having a plug positioned at a far end that is adapted to be inserted into a conventional electrical outlet 108. Electrical cord 150 enables locator unit 60 to receive power from the mains electrical supply via outlet 108. It will be appreciated that, in some embodiments, locator unit 60 is battery operated and cord 150 may be omitted. In still other embodiments, locator unit 60 may be both battery operated and include cord 150 so that in the event of a power failure, battery power supplies power to locator unit 60, and/or in the event of a battery failure, electrical power is received through outlet 108.

In some embodiments, locator units 60 include a video port that is adapted to receive a display cable 110 (FIG. 4). The display cable 110 is adapted to couple to locator unit 60 at one end and a display device 56 at its opposite send. Locator unit 60 may be configured to use cable 110 to send data to display device 56 that is to be displayed thereon. Such data may include data from one or more user devices 100 that are associated with the patient on patient support apparatus 20 (or with patient support apparatus 20 itself), data from one or more badges 142, status data from one or more sensors onboard patient support apparatus 20, location data regarding the location of patient support apparatus 20, and/or other data. Cable 110 may be a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. Locator unit 60 is configured to include a complementary type of connector that mates with a connector on an end of cable 110. In some embodiments, patient support apparatus 20, locator units 60, and display device 56 may be configured to display data in any of the manners disclosed in commonly assigned U.S. patent application Ser. No. 63/426,450 filed Nov. 18, 2022, by inventors Madhu Sandeep Thota et al. and entitled COMMUNICATION SYSTEM FOR PATIENT SUPPORT APPARATUSES, the complete disclosure of which is incorporated herein by reference.

In addition to the other functions described herein, locator units 60 are configured to communicate location data to patient support apparatus 20 that enables patient support apparatus 20 and/or patient support apparatus server 84 to determine the location of patient support apparatus 20 within the healthcare facility. In general, such location determination is carried out by patient support apparatus 20 analyzing wireless signals communicated between itself and locator unit 60 in order to determine its position relative to locator unit 60. If patient support apparatus 20, or a predefined reference point on patient support apparatus 20 (e.g. its head end, its center, etc.) is positioned within a threshold distance of locator unit 60, patient support apparatus 20 associates itself with the locator unit 60. When associated, patient support apparatus 20 may communicate data to locator unit 60, receive data from locator unit 60, and also deem its location within the healthcare facility to be the same as location of locator unit 60. When patient support apparatus 20 is outside of the threshold distance, it does not associate itself with locator unit 60, and therefore does not exchange data with locator unit 60 or consider its location to be the same as that of locator unit 60's location.

In some embodiments, patient support apparatus 20 is configured to associate itself with a particular locator unit 60 if controller 140 determines that the locator unit 60 is within a volume of space 152a (FIG. 4), or locator unit 60 determines that patient support apparatus 20 (or a reference point thereon) is positioned within volume of space 152a. In some embodiments, the volume of space 152a is defined with respect to each locator unit 60 and does not move. In other embodiments, the volume of space 152a is defined with respect to patient support apparatus 20 and moves as patient support apparatus 20 moves. In some embodiments, patient support apparatus 20 associates itself with a nearby locator unit 60 if both the locator unit 60 and the patient support apparatus 20 (or a reference point thereon) are concurrently within the predefined volume of space 152a. Regardless of whether volume of space 152a is defined with respect to a locator unit 60, or with respect to a patient support apparatus 20, by at least one or both of these devices (locator unit 60 and patient support apparatus 20) being positioned within the predefined volume of space 152a, the locator unit 60 and patient support apparatus 20 will be positioned within a threshold distance of each other. An example of patient support apparatus 20 occupying a volume of space 152a is shown in FIG. 4, where head end 38 of patient support apparatus 20 (that includes the appropriate reference point on patient support apparatus 20) is positioned inside of volume of space 152a, and patient support apparatus 20 has therefore associated itself with that particular locator unit 60.

After associating itself with a particular locator unit 60, patient support apparatus 20 is configured to be able to have its absolute position within the healthcare facility determined by receiving a unique locator identifier (ID) 122 (FIG. 5) from the locator unit 60. The location of each locator unit 60 in the healthcare facility is surveyed during the installation of locator units 60, and the unique IDs 122 of each locator unit 60 are also recorded during the installation of locator units 60. This surveying information and corresponding ID information may be stored in patient support apparatus server 84 and/or onboard the patient support apparatuses 20, thereby enabling a patient support apparatus 20 and/or patient support apparatus server 84 to determine the location of a patient support apparatus 20 once it is associated with a particular locator unit 60.

In those embodiments where patient support apparatus server 84 is configured to determine the location of patient support apparatus 20, patient support apparatus 20 sends its relative position information with respect to the associated locator unit 60, and/or the ID 122 of the associated locator unit 60 (and its own unique patient support apparatus ID 130 (FIG. 5)) to server 84. Server 84 includes a table of all of the locations of the locator units 60 (which, as noted, is generated via a surveying operation during the installation of locator units 60), and it uses that table to correlate the patient support apparatus IDs 130 and the locator unit IDs 122 it receives to specific locations within the healthcare facility. Thus, if a particular patient support apparatus 20 (with a particular ID 130) sends to server 84 an associated locator unit ID 122 that corresponds to room 430, server 84 determines that that particular patient support apparatus 20 is currently located in room 430. Generally speaking, and as will be discussed in greater detail below, the location of a patient support apparatus 20 is deemed to correspond to whichever locator unit 60 it is currently associated with, and if it is not currently associated with any locator unit 60, its location may be considered to be indeterminate.

In some embodiments of patient support apparatuses 20 and locator units 60, the relative location of a patient support apparatus 20 to a locator unit 60 is determined solely using ultra-wideband communication between the patient support apparatus 20 and the locator unit 60. Alternatively, in some embodiments, patient support apparatus 20 solely uses short range infrared communications with locator unit 60 to determine its relative location, wherein such short range infrared communications are only possible when the patient support apparatus 20 is positioned within a close proximity to the locator unit 60 (e.g. in the range of about 1-3 unobstructed meters). In these latter embodiments, patient support apparatus 20 may report that its location coincides with that of the nearby locator unit 60 when it is able to successfully communicate with the nearby locator unit 60 using these short range infrared communications. Still further, in some embodiments, patient support apparatus 20 and locator unit 60 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

In some embodiments, one or more user devices may interact with locator units 60 in the same or similar manner as the patient support apparatuses 20 interact with the locator units 60, as described above. That is, in some embodiments, locator units 60 may be configured to communicate location data to one or more user devices 100 that enables the user device 100, an associated patient support apparatus 20, and/or patient support apparatus server 84 to determine the location of the user device 100 within the healthcare facility. In general, such location determination is carried out by the user device 100 performing ultra-wideband ranging with the locator unit 60 in order to determine its position relative to locator unit 60. If the user device 100 is positioned within a threshold distance of locator unit 60, the user device associates itself with the locator unit 60. When associated, the user device 100 may communicate data to locator unit 60, receive data from locator unit 60, and also deem its location within the healthcare facility to be the same as location of locator unit 60. When user device 100 is outside of the threshold distance, it does not associate itself with locator unit 60, and therefore may cease to exchange data with locator unit 60.

FIG. 5 depicts a block diagram of patient support apparatus 20, a linked locator unit 60, a first user device 100a, a second user device 100b, network 80, and a plurality of electronic devices that the network 80 may be in communication with. As will be discussed in greater detail below, patient support apparatus 20 is configured to automatically determine the location of one or more locator units 60, badges 142, and/or user devices 100 that either have built-in UWB transceivers or a UWB tag attached to them. In addition, patient support apparatus 20 is configured to automatically carry out communications with these devices (including user devices 100) if they are positioned within a defined proximity to patient support apparatus 20. In some embodiments, if a particular user device 100 is positioned within the defined proximity, patient support apparatus 20 automatically associates the user device 100 with the patient assigned to patient support apparatus 20 (and/or with patient support apparatus 20 itself), and causes data from that user device 100 (or devices 100) to be automatically directed to one or more destinations. When the user device 100 is positioned outside the defined proximity, patient support apparatus 20 may automatically disassociate itself from the user device 100 and, in some situations, terminate communications with the user device 100 and/or inform patient support apparatus server 84 of the disassociation.

Linked locator unit 60 (FIG. 5) includes an ultra-wideband transceiver 126, a Bluetooth transceiver 106, a locator unit controller 112, configuration circuitry 114, a television controller 116, a headwall interface 118, a network transceiver 120, a unit ID 122, and, in some embodiments, an infrared transceiver 124. Bluetooth transceiver 106 is adapted to communicate with a Bluetooth transceiver 128 onboard patient support apparatus 20 using RF waves in accordance with conventional Bluetooth standards (e.g. IEEE 802.14.1 and/or any of the standards maintained by the Bluetooth Special Interest Group (SIG) of Kirkland, Washington, USA). In some embodiments, transceivers 106 and 128 utilize Bluetooth Low Energy communications.

Ultra-wideband transceiver 126 is adapted to communicate with one or more ultra-wideband transceivers 132 positioned onboard patient support apparatus 20 and/or one or more ultra-wideband transceivers 158, 178 positioned onboard user devices 100. Transceiver 126 is adapted to determine a distance between itself and patient support apparatus 20 and/or a user device 100. Alternatively, or additionally, transceiver 126 may be adapted to allow one or more of the UWB transceivers 132 onboard patient support apparatus 20 (or one or more of the UWB transceivers 158, 178 onboard the user device 100) to determine their distance(s) from transceiver 126. In other words, one or more location engines may be positioned onboard the patient support apparatus 20, onboard the locator unit 60, onboard the user devices 100, and/or onboard server 84.

In some embodiments, transceivers 126, 132, 158, and 178 use time of flight (TOF) computations to determine these distances. In other embodiments, transceivers 126, 132, 158, and 178 may utilize other techniques (e.g. time difference of arrival, two-way ranging, angle of arrival, channel state information, etc.) for determining their distances from each other, either in addition to, or in lieu of, TOF computations. In some embodiments, transceivers 126, 132, 158, and 178 may also determine an angle between themselves using angular information derived from antenna arrays positions onboard transceivers 126, 132, 158, and 178, or by using other techniques. The position and orientation of each transceiver 132 onboard patient support apparatus 20 is known and stored in an onboard memory 134 and used to determine the position and orientation of patient support apparatus 20 with respect to the locator unit(s) 60 with which it is communicating. Such position and orientation information may be determined using conventional trilateration and/or triangulation techniques, or other techniques.

In some embodiments, transceivers 126, 132, 158, and 178 are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 126, 132, 158, and 178.

Locator unit controller 112 is adapted to control the operation of transceivers 126, 106, configuration circuitry 114, TV controller 116, headwall interface 118, network transceiver 120, and, if included, IR transceiver 124 (FIG. 5). When infrared transceiver 124 is included, it may be included to provide backwards compatibility to patient support apparatuses 20 that are not equipped with a UWB transceiver 132. That is, some healthcare facilities may include one or more patient support apparatuses that are not equipped with a UWB transceiver 132, but that do include an IR transceiver that is adapted to communicate with IR transceiver 124. When locator unit 60 includes IR transceiver 124, it is able to communicate its unit ID 122 to such patient support apparatuses via IR transceiver 124, which is a short range transceiver that is configured to only communicate with an adjacent patient support apparatus when the patient support apparatus is nearby (e.g. without about five feet or so). Such an adjacent patient support apparatus 20 then communicates the received locator unit ID 122 along with its own unique ID 130 (FIG. 5) to server 84 which, as noted previously, is able to correlate the locator unit ID 122 to a particular location with the healthcare facility. In this manner, server 84 is able to use locator units 60 determine the location of versions of patient support apparatuses 20 that don't have a UWB transceiver 132, but that do have an IR transceiver.

Headwall interface 118 is adapted to change the electrical state of one or more pins that are in electrical communication with communication outlet 64 (via cable 66). Headwall interface 118 changes these electrical states in response to instructions from controller 112. For example, if the exit detection system 136 of patient support apparatus 20 detects a patient exit, a controller 140 of patient support apparatus 20 sends an exit alert signal to linked locator unit 60 and controller 112 responds by instructing headwall interface 118 to change the electrical state of at least one pin that is used to signal an exit alert (or a generic priority alert) to the nurse call system 70 via communications outlet 64. Additionally, if a device 100, such as a portable exit detection sensor, is associated with patient support apparatus 20 and it detects a patient exit, the exit detection sensor may transmit an exit detection alert signal to patient support apparatus 20, which in turn forwards the exit alert signal to linked locator unit 60, and controller 112 responds by instructing headwall interface 118 to change the electrical state of the same pin or pins that it does in response to receiving an exit detection alert from exit detection system 136.

In some embodiments, headwall interface 118 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 118 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 60 may also be configured to perform any of the functions of the headwall units 94 disclosed in the above-mentioned '778 patent application.

Configuration circuitry 114 and TV controller 116 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 60 may be configured to perform any of the functions of the headwall units 144 disclosed in the aforementioned '508 patent application.

Headwall interface 118, television controller 116, and configuration circuitry 114 may be omitted from unlinked locator units 60. This is because unlinked locator units 60 are not adapted to communicate with a communication outlet 64 and these components are designed for communications with outlet 64. Unlinked locator units 60 may also omit (or include) IR transceiver 124. Linked locator units and/or unlinked locator units 60 may optionally include (or omit) network transceiver 120. When included, controller 112 may use network transceiver 120 to communicate with patient support apparatus server 84 (via access points 82 and network 80).

Patient support apparatus 20 includes a controller 140, a memory 134, exit detection system 136, a scale system 144, monitoring system 138, a microphone 146, Bluetooth transceiver 128, one or more UWB transceivers 132, display 52 (which may be part of control panel 54a, and/or another control panel 54), network transceiver 96, a nurse call interface 154, and a plurality of additional components that are not shown in FIG. 5. Each UWB transceiver 132 is positioned at a known location on patient support apparatus 20. This known location information is stored in memory 134 and/or elsewhere, and may be defined with respect to any suitable frame of reference that is common to patient support apparatus 20. The known location information may include the spatial relationship between UWB transceivers 132 and/or any other components of patient support apparatus 20. For example, in some embodiments, the known location information includes the spatial relationship not only between UWB transceivers 132, but also the spatial relationships between UWB transceivers 132 and one or more of the following: the head end 38 of patient support apparatus 20, the foot end 40 of patient support apparatus 20, the sides of patient support apparatus 20, a reference point defined on patient support apparatus 20, the floor, and/or other components and/or landmarks of patient support apparatus 20. In some embodiments, this location information is used to determine the orientation of patient support apparatus 20 with respect to one or more walls 62, locator units 60, another patient support apparatus 20, and/or another object or structure within the healthcare facility.

In some embodiments, patient support apparatus 20 includes four UWB transceiver 132, each of which are positioned generally adjacent one of the four corners of patient support apparatus 20. In some such embodiments, the four UWB transceiver 132 are attached to, or positioned near, the four corners of litter frame 28. In other embodiments, the four UWB transceivers 132 are attached to, or positioned near, the four corners of base 22. In some embodiments, each of the four UWB transceivers 132 are attached to the corners of support deck 30. Still other locations of the UWB transceivers 132, as well as different numbers of the UWB transceiver 132, may be incorporated into patient support apparatus 20. In those embodiments of patient support apparatus 20 where one or more of the UWB transceivers 132 are coupled to components of patient support apparatus 20 that are movable (e.g. litter frame 28, which can have its height and orientation changed; or support deck 30 that can have its sections, such as head section 44, pivoted), sensors are included within patient support apparatus 20 that communicate the current position of the movable component to controller 140 so that controller 140 is able to determine the current positions of the UWB transceivers 132 and use those positions when determining the current location of a device, such as a badge 142 and/or another device 100.

Nurse call interface 154 of patient support apparatus 20 (FIG. 5) includes Bluetooth transceiver 128 and a cable port 148, in some embodiments. Nurse call interface 154 provides an interface for patient support apparatus 20 to communicate with outlet 64 of nurse call system 70. That is, nurse call interface 154 provides the means for patient support apparatus 20 to bidirectionally communicate with communication outlet 64. As has been discussed, in some situations, patient support apparatus 20 uses Bluetooth transceiver 128 to communicate with Bluetooth transceiver 106 of linked locator unit 60, and linked locator unit 60 forwards communications back and forth between outlet 64 and patient support apparatus 20. In other words, in some situations, linked locator unit 60 functions as a communications intermediary between nurse call interface 154 and outlet 64. Alternatively, a nurse call cable 66 may be coupled directly between patient support apparatus 20 and wall outlet 64, thereby avoiding the need to use linked locator unit 60 as a communication intermediary. In such situations, one end of a nurse call cable 66 is plugged into cable port 148 of patient support apparatus 20 and the other end of the cable 66 is plugged directly into outlet 64. Nurse call interface 154 thereby provides patient support apparatus 20 with the ability to communicate either wirelessly or via wired means with the outlet 64.

Patient support apparatus 20 also includes, in at least some embodiments, a microphone 146 (FIG. 5) that is used to detect the voice of the patient when the patient wants to speak to a remotely positioned nurse. The patient's voice is converted to audio signals by microphone 146 and controller 140 is adapted to forward these audio signals to an adjacent communications outlet 64 positioned in wall 62 (FIG. 4). When a cable 66 is coupled between cable port 148 of patient support apparatus 20 and outlet 64, controller 140 forwards these audio signals to outlet 64 via the cable 66. When no such cable 66 extends between patient support apparatus 20 and outlet 64, controller 140 wirelessly forwards these audio signals to the linked locator unit 60 that it is currently associated with (using transceiver 128, or in some embodiments, one of transceivers 132) and controller 112 of linked locator unit 60 forwards these audio signals to outlet 64. As was noted, outlet 64 is in electrical communication with a conventional nurse call system 70 that is adapted to route the audio signals to the correct nurse's station 78, and/or other location. In some embodiments, microphone 146 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

In the example shown in FIG. 5, there are two types of user devices 100a and 100b positioned in the vicinity of patient support apparatus 20. The first type of user device 100a is a mobile communication device, such as a cell phone, a tablet computer, or the like, used by the patient to communicate with people located outside of the room 58. The second type of user device 100b is a control device, such as a patient support apparatus pendant, used by the patient (or other individual) for controlling aspects of patient support apparatus 20. In some embodiments, as discussed above, the pendant user devices 100b may include a control panel of the type shown in FIG. 3.

User device 100a includes a controller 156, a UWB transceiver 158, a microphone 160, a speaker 162, a network transceiver 164, a unique ID 166, and at least one software application 168. User device 100b includes a controller 170, a Bluetooth transceiver 172, UWB transceiver 178, an identifier 176, and a plurality of controls 174. In some embodiments, the controls 174 of user device 100b correspond to one or more of the controls 50g-t of the control panel of FIG. 3.

The UWB transceivers 158, 178 of user devices 100a and 100b are adapted to communicate with the UWB transceivers 132 positioned onboard patient support apparatus 20 so that the position of the user devices (both 100a and 100b) relative to patient support apparatus 20 can be repetitively determined. The UWB transceivers 158, 178 of user devices 100a and 100b may be the same as all of the other UWB transceivers discussed herein (e.g. UWB transceivers 132, 126, etc.). The UWB transceiver 158 of user device 100a is further adapted to transmit unique ID 166 of user device 100a to patient support apparatus 20 so that patient support apparatus 20 knows which specific user device 100a it is communicating with. Similarly, the UWB transceiver 178 of user device 100b is further adapted to transmit unique ID 176 of user device 100b to patient support apparatus 20 so that patient support apparatus 20 knows which specific user device 100b it is communicating with.

It will be understood that the components of user devices 100a and 100b shown in FIG. 5 are merely illustrative examples of user devices 100, and that different user device 100 may be utilized with the system of the present disclosure that have fewer, greater, and/or different components than those shown in FIG. 5.

Controller 156 of user device 100a is adapted to oversee the operation of user device 100a, process the communications of UWB transceiver 158 with other UWB transceivers (e.g. transceivers 132), respond to the activation of controls on user device 100a (if any), oversee the operation of microphone 160 and speaker 162, and execute the software application 168. In some embodiments, as noted, user device 100a may be a conventional smart phone, a conventional tablet computer, or other conventional computer device that is adapted to execute software application 168. In other words, user device 100a, in some embodiments, is a conventional device with the exception of software application 168 which, when executed by controller 156, provides the user device 100a with new functionalities and interaction capabilities with respect to not only patient support apparatus 20, but also other devices within the healthcare facility.

User device 100a of FIG. 5 is adapted to allow a caregiver to speak into microphone 160 and have his or her voice transmitted to a remotely positioned phone, a remotely positioned caregiver badge 142, and/or another type of remotely positioned computing device. Similarly, user device 100a of FIG. 5 is adapted to receive audio signals from a remotely positioned phone, a remotely positioned caregiver badge 142, and/or another type of remotely positioned computing device, and to route them to speaker 162 so that the patient can hear those audio signals. In other words, user device 100a is adapted to allow a patient to communicate with remotely positioned personnel. Further, as will be discussed in greater detail below, software application 168 is adapted to automatically facilitate this remote communication without requiring the patient or any healthcare personnel to input a telephone number and/or other contact information into the user device 100a. This automatic facilitation is based upon a determination of the association status of user device 100a with respect to patient support apparatus 20.

Controller 170 of user device 100b (FIG. 5) is adapted to oversee the operation of the pendant user device 100b. As was noted previously, pendant user device 100b is adapted to allow a patient to control various operations of patient support apparatus 20. As was described previously, controller 170 may be configured to send motion commands, in response to a user's activation of one or more motion controls 174, to patient support apparatus 20 via both Bluetooth transceiver 172 and via UWB transceiver 178. In addition, controller 170 is configured automatically utilize UWB transceiver 178 to determine the relative position of pendant user device 100b relative to patient support apparatus 20. If pendant user device 100b is positioned inside of a predetermined threshold of patient support apparatus 20 (such as predetermined volume of space 152b), controller 170 forwards commands (both motion and non-motion commands) to patient support apparatus 20. If pendant user device 100b is positioned outside of the predetermined threshold of patient support apparatus 20, controller 170 does not forward commands (both motion and non-motion commands) to patient support apparatus 20. Stated alternatively, controller 170 uses UWB transceiver 178 to determine whether to associate itself with patient support apparatus 20 or not, and, if associated, it allows commands to be sent to patient support apparatus 20, and if not associated, it doesn't allow commands to be sent to patient support apparatus 20.

Controller 140, as well as controllers 112, 156, and 170 may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 112, 140, 156, and 170 when carrying out the functions described herein, as well as the data necessary for carrying out these functions, are stored in a corresponding memory that is accessible to that particular controller (e.g. memory 134 for controller 140). In some embodiments, controllers 112, 140, 156, and 170 may include and/or work with a microcontroller that is integrated into, or associated with, the UWB transceiver(s) aboard that particular device (e.g. UWB transceivers 126, 132, 158, and 178), and that microcontroller may act as a location engine, either alone or in combination with controller 112, 140, 156, and 170, for determining the locations of the other UWB transceivers with which it is in communication.

Controller 140 of patient support apparatus 20 utilizes UWB transceivers 132 to determine the relative position of patient support apparatus 20 with respect to one or more nearby locator units 60, one or more user devices 100, and/or one or more badges 142. In some embodiments, patient support apparatus 20 may also use UWB transceiver 132 to determine the relative position of other objects to patient support apparatus 20, such as one or more medical devices, or other types of devices. If patient support apparatus 20 is positioned within range of a locator unit 60, its UWB transceivers 132 communicate with the UWB transceiver 126 positioned on that locator unit 60, and the transceivers 132 and 126 exchange signals that enable them to determine the distances between themselves. This distance determination is done for each UWB transceiver 132 positioned onboard patient support apparatus 20 (or for as many as is necessary in order to determine an accurate position of locator unit 60 relative to patient support apparatus 20).

If a user device 100 is positioned within range of the UWB transceivers 132 of patient support apparatus 20, the UWB transceivers 158 and/or 178 of the user device 100 communicate with the UWB transceivers 132 of patient support apparatus 20 and exchange signals that enable them to determine the distances between themselves. In some embodiments, this distance determination is done for each UWB transceiver 132 positioned onboard patient support apparatus 20 (or for as many as is necessary in order to determine an accurate position of the user device 100 relative to patient support apparatus 20).

Similarly, if a caregiver badge 142 is positioned within range of the UWB transceivers 132 of patient support apparatus 20, the UWB transceiver(s) of the caregiver badge 142 communicate with the UWB transceivers 132 of patient support apparatus 20 and exchange signals that enable them to determine the distances between themselves. In some embodiments, this distance determination is done for each UWB transceiver 132 positioned onboard patient support apparatus 20 (or for as many as is necessary in order to determine an accurate position of the badge 142 relative to patient support apparatus 20).

In some embodiments, the UWB transceivers (126, 132, 158, 178, and the UWB transceivers integrated into badges 142) may also be configured to determine an angular relationships between themselves. The distance (and angle information) in at least some embodiments is calculated by UWB transceiver 132 and/or controller 140 of patient support apparatus 20. In other embodiments, one or more of the other device 60, 100, 142 may also, or alternatively, calculate the distance (and angle information) and forward the results of this calculation to patient support apparatus 20 (either via a UWB transceiver or BT transceiver). In either situation, patient support apparatus controller 140 is informed of the distances (and, in some embodiments, as noted, the angle information) between its UWB transceivers 132 and those onboard nearby device. These distances and orientations are then used to calculate a relative position of patient support apparatus 20 to these devices (locator unit 60, user device 100, and/or caregiver badge 142) in a common frame of reference that may be defined in a fixed relationship to the patient support apparatus 20 or the device.

Although FIGS. 4 and 5 only illustrate a single locator unit 60, it will be understood that a typical healthcare facility will include multiple locator units 60 positioned at different locations throughout the facility, including ones positioned within patient rooms and others positioned outside of patient rooms. Typically, at least one locator unit 60 will be positioned in each patient room of the healthcare facility, and if the patient room is intended to be occupied by more than one patient (e.g. it includes multiple bays), then additional locator units 60 may be included so that each patient support apparatus 20 will have a locator unit 60 positioned adjacent to each bay area in the room. Additional locator units 60, such as unlinked locator units 60a, may also be positioned at other locations through the healthcare facility.

The location of patient support apparatus 20 relative to locator units 60, user devices 100, and badges 142 is repetitively determined by an exchange of signals between their UWB transceivers 126, 132, 158, and 178. This exchange is initiated by an interrogation signal that may be sent by the UWB transceivers 126 of one or more of these devices (20, 60, 100, and/or 142). The trigger for sending these interrogation signals (from either source) may simply be the passage of a predefined interval of time, in at least some embodiments. That is, in some embodiments, patient support apparatus 20, locator units 60, user devices 100, and/or badges 142 may be configured to periodically send out an interrogation signal that will be responded to by any UWB transceivers that are positioned with range of that signal. In those embodiments where patient support apparatuses 20 are configured to send out such an interrogation signal, the time intervals between the interrogation signals may be varied depending upon the location, the number of user devices 100 and/or badges 142 (if any) that are positioned within range of patient support apparatus 20, and/or the status of the patient support apparatus 20. For example, in some embodiments, controller 140 may be configured to send out the interrogation signals with longer timer intervals between them when the patient support apparatus is stationary (and, in some cases, when no user devices 100 or badges 142 are currently positioned in communication range), and to send out the interrogation signals with shorter time intervals between them when the patient support apparatus 20 is in motion and/or when at least one device 100 or badge 142 is currently positioned within communication range of transceivers 132. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements and looking for changes indicative of movement.

In some embodiments, the UWB transceivers of each UWB device 20, 60, 100, and 142 are configured to act as either UWB anchors or as UWB tags. In at least one embodiment, the UWB transceivers 126 of locator units 60, the UWB transceivers 158, 178 of user devices 100, and the UWB transceivers of caregiver badges 142 are configured to all act as UWB tags, while the UWB transceivers 132 of patient support apparatus 20 are configured to act as UWB anchors. It will be understood that modifications to these roles of anchors and tags can be made. For example, in some embodiments, the UWB transceivers 132 of patient support apparatus 20 may be modified to act as UWB anchors in some instances and as UWB tags in other instances. Still other modifications can be made.

In general, when a UWB transceiver is configured to act as a UWB tag, it is configured to periodical transmit a UWB start packet, which acts as a discovery packet. The start packet requests that any UWB anchors that are within communication range to respond. If another UWB transceiver that is acting as a UWB tag happens to receive the start packet from another UWB tag, that UWB transceiver is configured to not respond to it. In other words, tags transmit start packets, but do not respond to start packets. UWB anchors, on the other hand, do not transmit start packets, but instead respond to start packets with a response packet that may be referred to as a stamp packet. Anchors therefore transmit stamp packets, but do not transmit start packets. UWB anchors also do not respond to other stamp packets that they may detect from other UWB anchors. In response to receiving a stamp packet from a UWB anchor, the UWB tags are configured to transmit an end packet back to the UWB anchor that transmitted the stamp packet.

The combination of the start, stamp, and end packet generally defines a ranging session between a UWB anchor and a UWB tag. The ranging session uses time of flight (TOF) information generated from the start, stamp, and end packets to allow the anchor and/or tag to determine a distance between the tag and the anchor. In some embodiments, the start, stamp, and/or end packet may also contain other data in their payloads that is used for other purposes besides ranging. From the ranging information, the distance between the anchor and tag is determined. These ranging sessions are repetitively carried out while a UWB anchor and UWB tag are within communication range.

In some embodiments, the time interval between ranging sessions is controlled by the UWB tag. That is, after the UWB tag sends a start packet, receives a stamp packet in response, and sends an end packet in response to the stamp packet, the UWB tag is configured to wait a defined amount of time before sending out another start packet. The defined amount of time is programmed into the UWB tag and can be varied during operation of the UWB tag. In some embodiments, the UWB tag may be configured to change this defined amount of time (hereinafter, the “ranging interval”) based upon whether the UWB device with the tag is associated with, or not associated with, a UWB device having an anchor. In other embodiments, the ranging interval may be changed by a tag based upon the status of one or more of the UWB devices that are involved in the ranging session. Such status may include, but is not limited to, the movement status of one or more of the UWB devices, the position and/or state of one or more components of the devices 250, the location of the device 250 within the healthcare facility and/or in relation to other UWB device(s), and/or other factors.

After the installation of locator units 60 in a particular healthcare facility, the location of each locator unit 60 within that facility is recorded. In some embodiments, the coordinates of the locations of locator units 60 are recorded in a common frame of reference (or converted to a common frame of reference after recordation). Such coordinates may be three dimensional (i.e. include a vertical and two horizontal components), or they may be two dimensional (no height component). In other embodiments, a more generalized location of one or more locator units 60 is determined, rather than the precise coordinates of the locator units 60. The generalized location of the locator units 60 may include an indication of the room, bay, area, hallway, portion of a hallway, wing, maintenance area, etc. that the locator unit 60 is positioned in. In still other embodiments, the locations of one or more locator units 60 are determined both generally and more precisely.

Regardless of how the location of each locator unit 60 is initially determined after they are installed in a healthcare facility (e.g. whether their coordinates are determined or a more generalized location is determined), the locations of all of the locator units 60, as well as their unique IDs 122, are stored in a memory accessible to server 84. Server 84 then uses this location data and ID data to determine the location of a patient support apparatus 20 (as well as the location of associated user devices 100 and badges 142). Alternatively, or additionally, the location data and IDs 122 are forwarded to patient support apparatuses 20 for storage in their onboard memories 134 and for use in determining their own locations. In some embodiments, the location of each locator unit 60 (whether specific and/or general) may also, or alternatively, be stored in a memory within that particular locator unit 60 and shared with the devices it communicates with (e.g. patient support apparatuses 20). In some other embodiments, the location of each locator unit 60 may be stored in multiple locations.

It will be appreciated that patient support apparatuses 20 are configured to communicate with locator units 60 regardless of the orientation of the patient support apparatus 20. That is, the UWB transceivers 126 and 132 are radio frequency transceivers that do not rely on line of sight communication, unlike the IR transceiver 124 (if present). Thus, the patient support apparatuses 20 do not have to be pointed in any particular direction with respect to the locator units in order for transceivers 126 and 132 to communicate. This differs from some prior art systems that use IR communication between the patient support apparatuses 20 and the locator units and that require the IR transceiver onboard the patient support apparatus to be aimed toward the locator unit in order for communication to be established. It will also be understood that locator units 60 can be positioned on walls, columns, ceilings, or any other fixed structures within the healthcare facility.

Patient support apparatus 20 may also be configured to use UWB transceivers 132 to repetitively determine the position of other devices relative to patient support apparatus 20, beyond the user devices 100, locator units 60, and/or caregiver badges 142. When so configured, controller 140 uses UWB transceivers 132 to determine the relative position of these other in the same manner discussed above for user devices 100, locator unit 60, and/or caregiver badges 142. These other devices may include, but are not necessarily limited to, any one or more of the following: another patient support apparatus 20, an infusion pump, a vital sign sensor, an exercise device, a heel care boot, an IV stand and/or pole, a ventilator, a DVT pump, a patient monitor (e.g. a saturated oxygen (SpO₂) monitor, an EKG monitor, a vital sign monitor, etc.), a patient positioning device (e.g. a wedge, turning device, pump), an ambient sensor (e.g. air temperature, air flow, light, humidity, pressure, altitude, sound/noise), a mattress 42, a portable exit detection sensor, an attachable nurse call device, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, a patient temperature management device (or associated device, such as a one or more hoses, thermal wraps, etc.), one or more mobility assistance devices that a patient may be expected to use, and/or still other types of user devices.

In those embodiments where patient support apparatus 20 is configured to perform UWB ranging with an infusion pump, patient support apparatus 20 and patient support apparatus server 84 may be configured to carry out any of the functions associated with the infusion pump that are described in commonly assigned U.S. patent application Ser. No. 63/349,369 filed Jun. 6, 2022, by inventors Krishna Bhimavarapu et al. and entitled COMMUNICATION SYSTEM FOR PATIENT SUPPORT APPARATUSES, the complete disclosure of which is incorporated herein by reference. In those embodiments where patient support apparatus 20 is configured to perform UWB ranging with a portable exit detection sensor, another patient support apparatus, and/or an attachable nurse call device, patient support apparatus 20 and patient support apparatus server 84 may be configured to carry out any of the functions associated with the portable exit detection sensors, nurse call devices, and secondary patient support apparatuses disclosed in commonly assigned U.S. patent application Ser. No. 63/352,061 filed Jun. 15, 2022, by inventors Jerald Trepanier et al. and entitled COMMUNICATION SYSTEM FOR PATIENT SUPPORT APPARATUSES, the complete disclosure of which is incorporated herein by reference.

For all of the UWB devices that patient support apparatus 20 is configured to determine the location of (i.e. perform UWB ranging with), controller 140 of patient support apparatus 20 uses the relative position information to determine how it will interact with these devices, including whether to associate with these devices or not. When controller 140 associates patient support apparatus 20 with one or more of these devices, as will be discussed in greater detail below, controller 140 and/or server 84 may take one or more of the following actions: display data from these devices on display 52 and/or another display device; automatically route data from one or more of these devices to one or more appropriate destinations, and/or take other actions; retrieve data about one or more of these devices from patient support apparatus server 84, EMR server 92, and/or badge sever 94 via network transceiver 96; send one or more signals from these devices to communication outlet 64 (via a cable 66 or through linked locator unit 60); forward one or more signals from outlet 64 to one or more of these devices; retrieve data from EMR server 92 that was generated by these devices; retrieve data from these devices via another route that is independent from EMR server 92; and/or perform other actions.

As discussed above, for locator units 60, controller 140 is configured to determine whether to automatically associate patient support apparatus 20 with a particular locator unit 60 based on whether both locator unit 60 and patient support apparatus 20 (or a reference point thereon) are positioned within a common volume of space (e.g. space volume 152a of FIG. 4). For user devices 100, controller 140 is configured to determine whether to automatically associate patient support apparatus 20 with a particular user device 100 based on whether the user device 100 is positioned within a predetermined volume of space that surrounds the patient support apparatus 20, such as space volume 152b of FIG. 4. For caregiver badges 142, controller 140 is configured to determine whether to automatically associate patient support apparatus 20 with a particular caregiver badge 142 based on whether the caregiver badge 142 is positioned within another predetermined volume of space that surrounds patient support apparatus 20, such as space volume 152c of FIG. 4. If patient support apparatus 20 is configured to associate with still other devices, controller 140 determines whether to associate with those other devices based upon whether those other devices are within one or more predetermined volumes of space that surround patient support apparatus 20, and such predetermined volumes of space may be the same as, or different from, one or more of the volumes of space 152 shown in FIG. 4 (and/or they may be different from each other).

Once a device—locator unit 60, user device 100, caregiver badge 142, etc.—is associated with patient support apparatus 20, it thereafter remains associated with patient support apparatus 20 until it moves outside of a particular volume of space, such as spaces 152a-c of FIG. 4, at which point controller 140 disassociates the device from patient support apparatus 20. That is, controller 140 repetitively determines and monitors the position of the devices while they are within UWB communication range, and if a device moves outside of a corresponding volume of space 152, controller 140 automatically disassociates the device from patient support apparatus 20. For example, controller 140 may make such an automatic disassociation if patient support apparatus 20 moves such that locator unit 60 is no longer inside space volume 152a (if space volume 152a is defined with respect to patient support apparatus 20), or such that patient support apparatus 20 is no longer inside space volume 152a (if space volume 152a is defined with respect to locator unit 60). Similarly, if a user device 100, for example, moves outside of a volume of space 152b defined around an associated patient support apparatus 20, controller 140 may automatically disassociate the user device 100 from the patient support apparatus 20.

In some embodiments, controller 140 may use modified volumes of space—such as, but not limited to, larger space volumes—when determining whether to automatically disassociate devices from patient support apparatus 20. In other words, once a device—such as a locator unit 60, a user device 100, and/or a caregiver badge 142—has been determined to be positioned inside of a particular volume of space, such as space volumes 152a-c (and any additional association conditions are met, if there are any), and controller 140 has associated the device with patient support apparatus 20, controller 140 may thereafter increase the size of—and/or otherwise change one or more dimensions of—the volume of space 152 when determining whether to automatically disassociate the device from patient support apparatus 20. In this manner, the volumes of space 152 may have a sort of hysteresis aspect wherein a device has to be positioned inside of a smaller space volume in order to be associated with patient support apparatus 20, but thereafter can only be disassociated if it moves outside of a larger sized volume of space. One example of this type of hysteresis effect is shown in FIG. 12 of commonly assigned U.S. patent application Ser. No. 63/356,242 filed Jun. 28, 2022, by inventors Madhu Sandeep Thota et al. and entitled PATIENT SUPPORT APPARATUS COMMUNICATION AND LOCATION SYSTEM, the complete disclosure of which is incorporated herein by reference. Controller 140 may be configured, in some embodiments, to utilize the hysteresis effect disclosed in the aforementioned '242 application, and/or to implement any of the functions of the patient support apparatuses disclosed therein. In still other embodiments, controller 140 may use the same dimensions for the volumes of space 152 for both association and disassociation purposes.

In some embodiments, space volumes 152a-c (FIG. 4) are defined with respect to patient support apparatus 20 and therefore move as patient support apparatus 20 moves. Space volume 152a, which may be the smallest of the space volumes 152, is generally used for the automatic association and disassociation between patient support apparatus 20 and a locator unit 60. Space volume 152b is generally used for the automatic association and disassociation between patient support apparatus 20 and user devices 100. Space volume 152c is generally used by controller 140 for the automatic association and disassociation between patient support apparatus 20 and badges 142. It will be understood that controller 140 may utilize other space volumes 152 than the three shown in FIG. 4.

Space volume 152c is generally sized such that it encompasses substantially all of the room 58 in which patient support apparatus 20 is positioned, or at least that portion of the room in which a healthcare worker with a badge 142 is expected to be present, particularly while the worker attends to the patient assigned to patient support apparatus 20 and/or to patient support apparatus 20 itself. In some situations, space volume 152c may be large enough to extend into adjacent rooms 58, but this will not affect the proper association of a badge 142 with patient support apparatus 20 so long as access to those rooms cannot be obtained without the healthcare worker exiting from space volume 152b before traveling to those other rooms. In other words, space volume 152c may be advantageously defined such that, when the person's badge 142 is moved out of room 58 through a doorway, the badge 142 will necessarily move outside of space volume 152c, thereby causing it to become disassociated from the patient support apparatus 20.

In some embodiments, any of space volumes 152a-c (FIG. 4) may be defined with one or more static dimensions. In other embodiments, patient support apparatus 20 and/or patient support apparatus server 84 may be configured to allow authorized individuals to change one or more dimensions of space volumes 152a-c. Still further, in some embodiments, space volume 152a-c may have variable dimensions based upon the specific room, bay, or other location, in which patient support apparatus 20 is currently positioned. In these embodiments, controller 140 may utilize a table stored in memory 134 that defines the dimensions of any of space volumes 152a-c based on the current location of patient support apparatus 20. The location of patient support apparatus 20 may be determined by controller 140 from the locator unit ID 122 that it receives from an associated locator unit 60, and/or it may be derived from information received from patient support apparatus server 84.

It will be understood that the dimensions of any of space volumes 152 may be variable, customizable, location dependent, and/or different from what is shown in FIG. 4. It will also be understood that, although the accompanying drawings all depict all of the space volumes 152 as having rectangular shapes, these shapes may be varied, including shapes that are all curved and/or shapes that have a combination of curved and straight boundaries. For example, in some embodiments, such as where patient support apparatus 20 has only a single UWB transceiver 132, the space volumes 152 may be spherical.

In some embodiments, controller 140 may be configured to allow a user to associate a user device 100 to patient support apparatus 20 via a manual process, in addition to the previously described automatic association process. For the manual process, the caregiver has to manually inform patient support apparatus 20 that a particular user device 100 should now be associated with that patient support apparatus 20. This manual process may be accomplished in different manners. For example, in some embodiments, the user device 100 and patient support apparatus 20 may include near field transceivers that, when positioned within close proximity (e.g. several inches) of each other, exchange information that establishes that that particular user device 100 should be associated with that particular patient support apparatus. Further details regarding the use of near field transceivers for associating user devices 100 to a patient support apparatus 20 are disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which has already been incorporated herein by reference.

Other types of manual association processes may also, or alternatively be used. For example, in some embodiments, if a user device 100a is a conventional cell phone, it can be manually associated with patient support apparatus 20 by connecting a conventional USB cable between the user device 100a and patient support apparatus 20. As another alternative, if user device 100a is a conventional cell phone, when the cell phone's UWB transceiver(s) 158 detects it is within a vicinity of a patient support apparatus 20, software application 168 may be configured to automatically display a message on a display of the cell phone asking the user (such as the patient) if he/she wants to pair their phone to patient support apparatus 20. Such pairing may then be accomplished by controller 140 displaying a 4-digit code (or a code with a different number of digits) on a display of patient support apparatus 20 (e.g. display 52) which the user then is asked to type into his or her cell phone to complete and verify the pairing (and association) process. Alternatively, controller 140 may be configured to audibly announce the code via one or more speakers onboard patient support apparatus 20, as well as to audibly instruct the user to enter the code into his or her cell phone. Controller 140 may also, or alternatively, blink one or more lights on patient support apparatus 20 and software application 168 will ask the user to confirm on his/her phone that the patient support apparatus 20 with the blinking lights is the one that he/she wishes to pair with. Still other types of manual association processes may also be used (some of which are also disclosed in the aforementioned '061 publication).

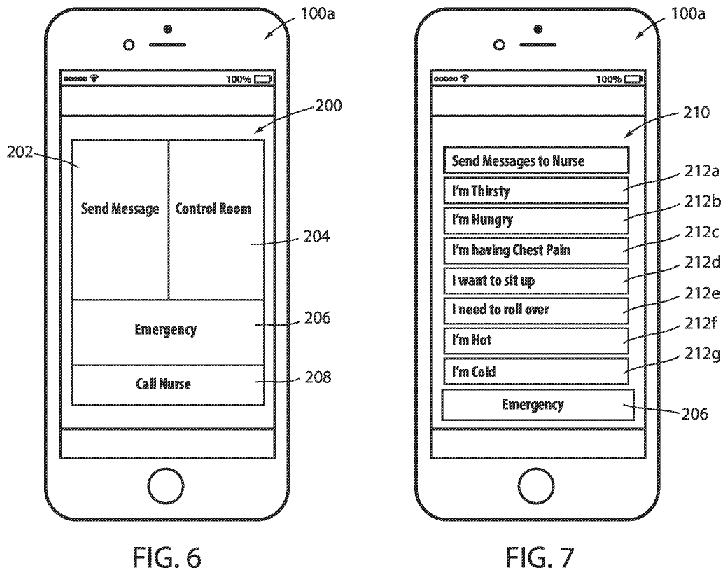

In some embodiments, when user device 100a is a conventional cell phone, a user may need to install software application 168 on the cell phone in order for it to be able to automatically associate with patient support apparatus 20. Alternatively, in some embodiments, when user device 100a is a cell phone, the cell phone may include the native ability to pair with UWB devices, and controller 140 may be able to associate itself with the cell phone without the installation of software app 168. However, regardless of whether or not software app 168 is required for association of the cell phone with patient support apparatus 20, software app 168 is configured to allow the user (e.g. the patient) to more easily communicate with the caregiver(s) who are assigned to that particular user, as will be discussed in more detail below with respect to FIGS. 6-10.

A user may install software app 168 by any conventional means, such as by navigating to the Google Play store, the Apple Store, or a similar store, depending upon whether the cell phone is Android based, Apple based, or built with another platform. In some embodiments of patient support apparatus 20, controller 140 may be configured to display information to the user facilitating the installation of software app 168 on the user's device. Such information may include a QR code, a URL, and/or other information for downloading software app 168. Indeed, in some embodiments, patient support apparatus 20 may store a copy of the app 168 and transfer it directly to the user device 100a.

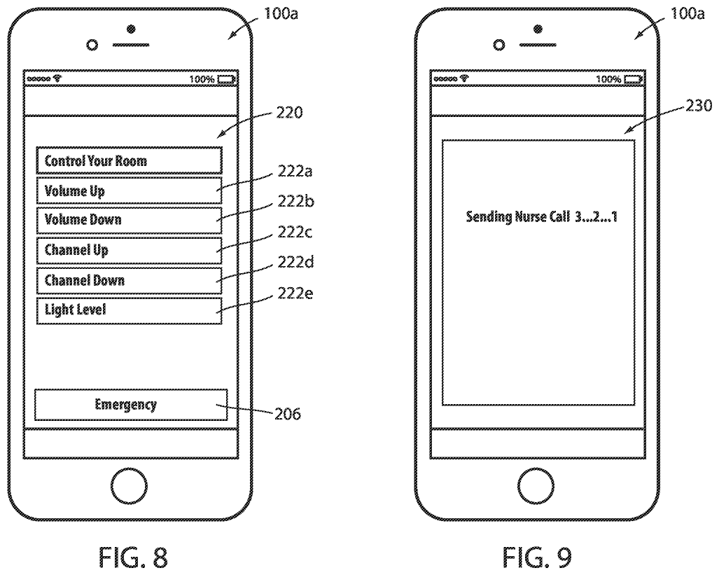

Once software app 168 (FIG. 5) has been installed on user device 100a, software app 168 is configured to provide the user with an improved ability to communicate with one or more remotely positioned caregivers. In some embodiments, software app 168 is configured to display a home screen, such as home screen 200 shown in FIG. 6. Home screen 200 includes a message sending option 202, a room control option 204, an emergency call option 206, and a non-emergency call option 208. In response to a user touching, or otherwise activating, message sending option 202, software app 168 is configured to cause controller 156 to display a message screen, such as the message screen 210 of FIG. 7. In response to a user touching, or otherwise activating, room control option 204, software app 168 is configured to cause controller 156 to display a room control screen, such as the room control screen 220 of FIG. 8. In response to a user touching, or otherwise activating, emergency call option 206, software app 168 is configured to cause controller 156 to display an emergency call screen, such as the emergency call screen 230 of FIG. 9. In response to a user touching, or otherwise activating, non-emergency call option 208, software app 168 is configured to cause controller 156 to automatically make a phone call to the caregiver assigned to the patient associated with user device 100a.

Message screen 210 (FIG. 7) includes a plurality of messages 212a-g that a user may select from to send to his or her assigned caregiver. These messages 212 include an “I'm thirsty” message 212a, an “I'm hungry” message 212b, an “I'm having chest pain” message 212c, an “I want to sit up” message 212d, an “I need to roll over” message 212e, an “I'm hot” message 212f, and an “I'm cold” message 212g. It will, of course, be understood, that the specific messages 212a-g that are shown in FIG. 7 may be varied. When a user touches, or otherwise activates, one of these messages 212a-g, software app 168 is configured to instruct controller 156 to send a message to the particular caregiver(s) assigned to care for the particular patient associated with that user device 100a (e.g. cell phone). As will be discussed in greater detail below, controller 140 and/or patient support apparatus server 84 are configured to automatically determine who the caregiver(s) are who are assigned to that particular patient, and to automatically route the message(s) from that particular patient's cell phone to the appropriate caregiver(s).

Room control screen 220 (FIG. 8) includes a plurality of room control options 222a-222e. Each room control option 222 allows the user (e.g. the patient) to control different aspects of the room 58 in which they are positioned. Volume up room option 222a causes the volume of a television 72 within the room to increase. Volume down option 222b causes the volume of the television 72 to decrease. Channel up option 222c causes the television 72 to change channels by switching to the next higher channel. Channel down option 222d causes the television 72 to change channels by switching to the next lower channel. Light level option 222e causes the light intensity of room light 74 to change (e.g. it may proceed in a looping fashion whereby its brightness increases and increases and then turns off, and then increases again). In some embodiments, room control screen 220 may include a separate option 222 for turning on the room light 74, a separate option 222 for turning off the room light 74, and/or a separate option for controlling the brightness. Additional control options may also be included for controlling reading light 76.

Emergency call screen 230 (FIG. 9) includes a single message indicating that an emergency call has been placed to the patient's caregiver. The user does not need to take any additional actions to implement this emergency call. Software app 168 is configured to make this phone call in response to the user selecting the emergency call option 206. Although not shown in the attached drawings, software app 168 may be configured to cause controller 156 to display a separate non-emergency call screen (not shown). Otherwise, software app 168 may be configured to make a non-emergency call to the patient's caregiver in response to the user selecting non-emergency option 208 (FIG. 6) without displaying a separate screen. In some embodiments, the difference between an emergency call and a non-emergency call is the recipient, or recipients, to whom the call is directed. For example, an emergency call may be, in some embodiments, directed to multiple people such that a call is established with whichever one of the multiple people answer first, while a non-emergency call may be directed to only a signal person. Other differences between emergency and non-emergency calls may also, or alternatively, be implemented. In some embodiments, there may be no difference between the two type of calls, and software app 168 may be modified to display only a single (emergency and non-emergency) calling option on home screen 200.

In response to a user selecting any of the options 222 on screen 220 (FIG. 8), software app 168 is configured to forward a room control message to patient support apparatus 20 using UWB transceiver 158 (although, in some embodiments, user devices 100a may include a Bluetooth transceiver, in which case controller 156 may forward the message using the Bluetooth transceiver). The content of the room control message corresponds to the content of the particular room control option 222 that was selected by the user. In order for controller 156 to send this room control message to patient support apparatus 20, controller 156 must first be associated with patient support apparatus 20. The association process provides controller 156 with an address, or other identification (e.g. patient support apparatus ID 130) that uniquely identifies the associated patient support apparatus 20 and allows controller 156 to specifically address the room control message to the particular patient support apparatus 20 with which it is associated. In other words, any other patient support apparatuses 20 that are positioned within communication range will ignore the room control message because it is not addressed to them.

After controller 140 of patient support apparatus 20 receives the room control message, it verifies that the message came from an associated user device 100a (based on, for example, the ID 166 contained within the message). After verifying it is from an associated user device 100a, controller 140 responds to the message in the same manner that it would respond to the activation of a corresponding room control 50 integrated into patient support apparatus 20 (or a corresponding control 50 from a corresponding pendant). For example, controller 140 responds to a volume up room control message from user device 100a in the same manner that it responds to the activation of volume up control 50l (FIG. 3) on control panel 54c (or pendant 100b). Controller 140 responds to a volume down room control message from user device 100a in the same manner that it responds to the activation of volume down control 50m on control panel 54c (or pendant 100b). Controller 140 responds to channel up and channel down room control messages from user device 100a in the same manners that is responds to the activation of channel up control 50n and channel down control 500, respectively, on control panel 54c (or pendant 100b). Similarly, controller 140 may respond to a light level room control message from user device 100a in the same manner that it responds to the activation of room light control 50t on control panel 54c (or pendant 100b).

In response to a user selecting any of the message options 212 on screen 210 (FIG. 7) or the call options 206, 208 on home screen 200 (FIG. 6), software app 168 may be configured to take different actions in different embodiments. In a first embodiment, where user device 100a is a cell phone or a tablet computer with cellular service, software app 168 instructs controller 156 to use its cellular service to send a text (corresponding to one of options 212a-g) to the patient's caregiver, or to use its cellular service to make a phone call (in response to options 206 or 208) to the patient's caregiver. In a second embodiment, where use device 100a is a cell phone or tablet computer with cellular service, software app 168 may be instructed to send a text message and/or make a phone call without using the cellular service. In the first embodiment, software app 168 automatically provides the phone number of the caregiver's cell phone to the patient's cell phone (or tablet computer) such that the patient does not have to enter this number into his or her cell phone. Software app 168 also automatically updates this phone number during caregiver shift changes (or other changes in caregiver work assignments) so that the patient's texts and/or phone calls will automatically be sent to the correct caregiver. In the second embodiment, software app 168 does not need to know the actual phone number of the caregiver's cell phone because the call and/or texts are routed to their intended destinations without using the cellular network.

Turning to the first embodiment, the phone numbers of the caregivers' cell phones may be maintained in a server of network 80, such as, for example, caregiver assignment server 104, or another server. Regardless of which specific server they are stored in, patient support apparatus server 84 is configured to retrieve these phone numbers. As was previously described, patient support apparatus server 84 is also configured to determine automatically which caregivers are assigned to which patients using communication with one or more of caregiver assignment server 104, ADT server 102, and/or EMR server 92. After patient support apparatus 20 has determined which caregiver is currently assigned to which patient and/or which caregiver is currently assigned to which room 58 within the healthcare facility, it sends the phone number of the assigned caregiver to the patient support apparatus 20 that is assigned to one of that caregiver's patients. In other words, after patient support apparatus server 84 determines that, for example, caregiver A is assigned to patient B, that patient B is assigned to room X, and that patient support apparatus Y is positioned in room X, it may be configured to automatically send the phone number of caregiver A's cell phone to patient support apparatus Y. Once patient support apparatus Y has this particular phone number, it automatically shares it with (and updates it with) user device 100a after user device 100a has become associated with patient support apparatus 20 (and stops sharing it with, and ceases to update it with, user device 100a after user device 100a has become disassociated with patient support apparatus 20). In this manner, software app 168 may be automatically provided with the correct phone number of the caregiver assigned to that particular patient, and this phone number may be automatically updated when a shift change, or other caregiver assignment change, occurs.

Turning to the second embodiment, software app 168 is configured to send a text request or phone call request to its associated patient support apparatus 20 in response to the patient selecting a message option 222 or a call option 206 or 208, respectively. If a user device 100a is not associated with a patient support apparatus 20 when a patient selects message option 222 or call option 206, 208, then software app 168 won't send a text request of phone call request to a patient support apparatus 20. After an associated patient support apparatus 20 receives the text request and/or phone call request message, it responds by forwarding the message to patient support apparatus server 84 using its network transceiver 96. The forwarded message includes the locator ID 122 of the locator unit 60 associated with that patient support apparatus 20, and/or the message includes the patient support apparatus ID 130 for that patient support apparatus 20. Patient support apparatus server 84 is configured to automatically determine where to route the phone call request or text request messages based on the particular location of the patient support apparatus 20 within the healthcare facility and data correlating that particular location to one or more particular caregiver phones, badges 142, and/or other destinations.

Thus, with reference to FIG. 5, after patient support apparatus 20 forwards a text request or phone call request message to patient support apparatus server 84 on local area network 80, patient support apparatus server 84 is configured to route those message(s) to a number of potential different destinations, including one or more non-interactive displays 240a, one or more interactive displays 240b, one or more caregiver badges 142, one or more caregiver phones and/or computers 242, and/or to nurse call system 70. Server 84 is configured to route these messages to one or more different destinations based upon its communication with one or more other servers on network 80 and/or based on different customization settings that may be implemented by authorized personnel at the healthcare facility.

In general, server 84 is configured to automatically determine which particular patient each particular user device 100a is associated with, and from that information automatically determine which caregiver is assigned to that particular patient, and from that information to automatically determine what device(s) (e.g. badges 142, caregiver cell phones 242, displays 242, etc.) are to receive information for that particular caregiver's patient. Server 84 then routes the phone call request or text request messages to that particular device (or devices).

Server 84 determines where to automatically route the phone call or text request message by gathering data from one or more additional servers on network 80. For example, server 84 is configured to receive patient-to-room correlation data from ADT server 102, and such data indicates the specific rooms that specific patients are located in. Server 84 is also configured to receive caregiver-to-room correlation data, or caregiver-to-patient correlation data from caregiver assignment server 104 (which may be a part of the nurse call system 70, in some embodiments), and such data indicates the specific caregiver(s) that are assigned to specific patients and/or to specific rooms. Server 84 is also configured to receive caregiver-to-badge correlation data from badge server 94, and such data indicates the specific caregiver associated with each specific badge 142. As noted, server 84 may be configured to receive caregiver-to-phone correlation data from another server, such as badge server 94 or another server on network 80, and such data indicates the specific phone (including phone number) associated with each specific caregiver.

In addition to the aforementioned data from other servers on network 80, patient support apparatus server 84 is further configured to receive caregiver-to-display or room-to-display correlation data from authorized personnel of the healthcare facility. Such data may be forwarded to server 84 by using a conventional computer that has access to network 80 and that is authorized to communicate with server 84. The caregiver-to-display or room-to-display correlation data indicates what specific displays 240 are associated with specific rooms within the healthcare facility or with specific caregivers.

In some embodiments, patient support apparatus server 84 is configured to determine patient-to-room, patient-to-bed, patient-to-bed-bay, patient-to-caregiver, caregiver-to-room, caregiver-to-patient-support-apparatus, and/or caregiver-to-bed-bay correlations in any of the manners disclosed in commonly assigned U.S. patent application Ser. No. 62/826,097, filed Mar. 29, 2019 by inventors Thomas Durlach et al. and entitled PATIENT CARE SYSTEM, the complete disclosure of which is incorporated herein by reference.

By using all of the aforementioned correlation data, patient support apparatus server 84 is configured to automatically determine what devices to route incoming text request and/or phone call request messages to. Server 84 then forwards the request messages to appropriate devices. Thus, for example, if a patient sends a text request message, that text request message may be automatically routed to the badge 142 associated with the specific caregiver assigned to that particular patient. If the healthcare facility doesn't use badges 142, or if the healthcare facility wants to configure server 84 differently, server 84 can be customized to, for example, send the text request message to the cell phone 242 of the specific caregiver assigned to that particular patient. Alternatively, or additionally, the healthcare facility can customize server 84 such that the text request message gets displayed on one or more displays 240. That is, patient support apparatus server 84 is configurable by authorized personnel at the healthcare facility to control where patient's text request messages (and phone call request messages) are routed within the healthcare facility.

After transmitting a phone call request message, software app 168 may be configured to instruct controller 156 to communicate the subsequent audio signals from the patient's voice (as generated from microphone 160) to the intended recipient using conventional Voice over IP (VOIP) communication protocols. In some embodiments, the VoIP signals are transmitted from user device 100a to patient support apparatus 20 (using either UWB and/or Bluetooth communications), and from patient support apparatus 20 to network 80 (e.g. server 84) using WiFi communications, and from network 80 to the intended recipient using WiFi communications and/or Ethernet communications.

Software app 168 makes it easy and convenient for a patient to use his or her own personal phone 100a (or tablet, or the like) for communicating with their caregiver(s), even when there is a shift change for the caregivers. Software app 168 allows the patient to communicate with the caregiver without requiring the patient to enter the caregiver's phone number, badge ID, or any other information into their personal phone 100a. Instead, once the patient brings his or her phone 100a inside of the predetermined volume of space 152b of their patient support apparatus 20, software app 168 communicates with patient support apparatus 20 and patient support apparatus server 84 to determine what caregiver is currently assigned to that particular patient (or room). Further, patient support apparatus server 84 automatically determines the phone number, badge ID, and/or other information necessary to communicate with that particular caregiver. From then on, as long as the patient's phone 100a remains associated with the patient's patient support apparatus 20, any texts or calls that the user places using his or her phone 100a will be route to network 80 and automatically forwarded to the correct caregiver's device 242, badge 142, and/or or display 240. Further, because patient support apparatus server 84 is in near real-time communication with caregiver assignment server 104 and/or other servers on network 80, if there is a shift change, server 84 is notified of the change and automatically adjusts where the patient's calls and/or texts are routed.

It will be understood that the automatic routing of the patient's calls and/or text to the appropriate caregiver that has been described above is different from conventional nurse call systems currently in existence. In such conventional systems, when a user presses on a button, or other control, on patient support apparatus 20, or a pendant connected thereto, such as control 50g (FIG. 3), patient support apparatus 20 sends a signal to communications outlet 64 which, in turn, forwards the signal over conductors 68 to a conventional nurse call system. Audio signals are then communicated back and forth between the nurse call system 70 and the patient support apparatus 20 over conductors 68 and through outlet 64. In many conventional healthcare facilities, this communication is only half-duplex, and the audio quality may be poor. Further, this communication is only operable if a nurse call cable 66 is properly connected to outlet 64 and either patient support apparatus 20 or locator unit 60. The system of the present disclosure, in contrast, not only provides full-duplex communications, but also allows the patient to send text messages, does not require any cables to be plugged in, and allows the patient to communicate using his or her own personal user device 100a (e.g. phone, tablet, etc.)

In some embodiments, patient support apparatus 20 may omit its conventional nurse call interface 154 (FIG. 5) because, as noted, the patient is able to communicate with his or her caregiver using a user device 100a. In other embodiments, patient support apparatus 20 may include both nurse call interface 154 and the ability to communicate with a user device 100a, thereby giving the patient potentially two different manners of communicating with his or her caregiver. Still further, in some embodiments, controller 140 may be configured to utilize both communications methods in certain situations (e.g. emergency calls). That is, for example, if the user presses the emergency call option 206 (FIG. 6) on user device 100a, controller 140 may be configured to forward the user's call bot to patient support apparatus server 84 using network transceiver 96 and to nurse call system 70 using nurse call interface 154.

Displays 240 (FIG. 5) may come in a variety of different forms. Some display 240 may be mobile display devices while other displays 240 may be stationary displays that generally remain in one location. Mobile displays may include laptop computers, Computers on Wheels (CoWs), and others. Stationary displays may include large screen smart televisions (e.g. with WiFi or network capability), personal computer displays, and others. The stationary displays 240 may be associated with a particular unit of a healthcare facility, a particular nurse's station, wing, floor, and/or other section of the healthcare facility. This information is provided to patient support apparatus server 84 so that server 84 can automatically determine whether to display information from a particular patient on a particular display. As was noted, in some embodiments, patient support apparatus server 84 is customizable by authorized individuals of the healthcare facility as to what information is displayed on displays 240. Thus, for example, if a patient sends a text “I'm thirsty,” the healthcare facility can choose whether this text will be automatically displayed on one or more displays 240, or whether it will simply be forwarded to a particular caregiver's (or caregivers′) phone/tablet 242, badge 142, or other device.

In some embodiments, displays 240, whether stationary or mobile, may communicate with patient support apparatus server 84 via a conventional web browser. It will be understood, however, that in other embodiments, some or all of the displays 240 may be modified to execute a specialized or native software application that is downloaded to the display and that is tailored to be executed by the particular operating system of the display.

In some embodiments, controller 140 may be configured to display data from a user device 100a associated with patient support apparatus 20, and/or process the data from the associated device, in any of the manners disclosed in commonly assigned U.S. patent application Ser. No. 63/306,279 filed Feb. 3, 2022, by inventors Madhu Sandeep Thota et al. and entitled COMMUNICATION SYSTEM FOR PATIENT SUPPORT APPARATUSES; or in commonly assigned U.S. patent application Ser. No. 63/426,450 filed Nov. 18, 2022, by inventors Madhu Sandeep Thota et al. and entitled COMMUNICATION SYSTEM FOR PATIENT SUPPORT APPARATUSES, the complete disclosures of which are both incorporated herein by reference.

In some embodiments, when a user devices 100b is a pendant type of user device, the pendant user device 100b may be configured to provide feedback to the user regarding the relative proximity of the pendant to patient support apparatus 20. For example, if the pendant 100b is moved beyond a threshold distance from patient support apparatus 20 (e.g. an outside a particular volume of space 152), the pendant may be configured to vibrate and/or flash one or more lights. Patient support apparatus 20 may also, or alternatively, be configured to flash one or more lights and/or emit an aural warning. The light flashing, vibration, and/or aural warning provides a warning to the caregiver and/or patient that the pendant is moving so far away from the patient support apparatus 20 that it will soon become disassociated from the patient support apparatus 20. In some embodiments, an additional and/or different warning may be provided when the pendant 100b is actually moved so far away from patient support apparatus 20 that it become disassociated from that patient support apparatus 20.

In some embodiments, when a pendant is moved close enough to a patient support apparatus 20 to initially associated with the patient support apparatus, the pendant 100b and/or the patient support apparatus 20 may be configured to flash one or more lights, vibrate, and/or emit an aural indication so that the user knows that the pendant 100b has become associated with that particular patient support apparatus 20.

Because each patient support apparatus 20 is repetitively attempting to range with any UWB devices that are within communication range (such as user devices 100) and because it is repetitively updating its association or disassociation status with any of those UWB devices that are within communication range, any pendant user device 100b may be used by a caregiver as a universal pendant. That is, a single pendant 100b can be carried by the caregiver from room to room and the pendant will automatically associate with the patient support apparatus 20 upon the caregiver's entry into the room (or other proximity to the patient support apparatus 20), and automatically disassociate from the patient support apparatus upon the caregiver's departure from the room (or other proximity of the patient support apparatus 20). When associated with a particular patient support apparatus 20, commands from the pendant 100b will only be directed to that particular patient support apparatus 20, and not to other patient support apparatuses 20. In this manner, the pendant 100b will automatically switch to controlling only the patient support apparatus 20 that is in close proximity to the caregiver, thereby enabling him/her to use a single pendant 100b for controlling each of the patient support apparatuses 20 of the patients they are assigned to care for.

FIG. 10 illustrates a communication diagram of several components of the patient support apparatus system of the present disclosure. More specifically, it illustrates one example of several types of communication that may take place between patient support apparatus 20, a patient's user device 100a, server 84, and a caregiver's phone/tablet 242 and/or badge 142. As shown therein, in some embodiments, a caregiver may use his/her device 242 and/or badge 142 to log into server 84 at step 250 and, in response, receive his or her assigned locations and/or patients (step 252). Step 254 refers to the automatic association process between the user's device 100a and patient support apparatus 20, and step 256 refers to the sending of this association status to server 84. In some embodiments, server 84 may be configured to automatically send a notification to the caregiver's phone/tablet 242 and/or badge 142 at step 258 if the patient moves to a new location (e.g. moves to the restroom, moves outside of his/her room, exits patient support apparatus 20, etc.). Such movement may be detected through the movement of the patient's user device 100a (although the exiting from the patient support apparatus 20 may also, or alternatively, be detected by exit detection system 136).

Step 260 (FIG. 10) illustrates a process where commands from the patient's phone 100a are sent to patient support apparatus 20 and acted upon by patient support apparatus 20 (e.g. any of room control options 204, commands to move components of patient support apparatus 20, and/or other commands). In some embodiments, data identifying these commands are sent at step 262 to server 84 and server 84 records this data. In this manner, server 84 builds a database of patient commands that are used with each patient support apparatus 20. The database can be used for diagnostic purposes, service purposes, and/or designing future improvements to software app 168, patient support apparatus 20, and/or server 84.

Step 264 illustrates the previously described process where data from the patient's phone 100a is sent to patient support apparatus 20 (step 264) and forwarded to server 84 (step 266). This data may include texts, audio calls, and/or other data. At step 268, server 84 routes the data to the appropriate destination, such as one or more caregiver phones/tablets 242, badges 142, etc. In some embodiments, server 84 may record the data it receives at step 266 (and/or characteristics of that data). The data may then be analyzed by server 84 to determine patterns for specific patients and/or which requests (text) are the most/least common, etc. In some embodiments, server 84 records the number of messages sent between patients and caregivers, response times, heat maps of travel, and/or other information.

In some embodiments, patient support apparatus 20 and/or server 84 may be configured to monitor and record the movement of the patient (via his/her phone 100a). This is illustrated in steps 270, 272, and 274 of FIG. 10. At step 270, the location of the caregiver's phone 100 is sent to patient support apparatus 20. This location information may be determined using UWB transceivers, or it may use other location-determining technology. At step 272, patient support apparatus 20 forwards the location of the patients' phone 100a to server 84, which in turn forwards this location to the appropriate caregiver's badge 142 or phone/tablet 242 at step 274. Server 84 may also record this location information to thereby generate a record of all of the locations to which a patient has traveled while in the healthcare facility.

Although the foregoing written description has focused on communicating data between one or more user devices 100a and patient support apparatus server 84 using patient support apparatus 20 as a communication intermediary, it will be understood that this may be modified, in some embodiments, such that an associated locator unit 60 functions as the communication intermediary, either in addition to, or in lieu of, the patient support apparatus 20. In such embodiments, a phone call from a patient's user device 100a may be routed from the user device 100a to an associated locator unit 60, from that locator unit 60 to network 80, and from network 80 to a badge 142, caregiver phone/tablet 242, and/or a display 240. The caregiver's audio signals are routed back to the user device 100a in the same path.

It will also be understood that, in some embodiments, any of the functionality of software app 168 may be built into patient support apparatus 20 itself, thereby avoiding the need for a separate user device 100a to be associated with the patient support apparatus 20. In such embodiments, patient support apparatus 20 may be configured to display any one or more of the options 202, 204, 206, and/or 208 on its own display 52 (or another display) and implement the same functionality for these functions as has been described herein.

In some embodiments, UWB transceivers 125, 132, 158, and 178 (FIG. 5) may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference. In some embodiments, locator units 60 may also be configured to determine the location of a device (e.g. user device 100, badge 142) in any of the manners disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

Although the foregoing description has primarily indicated that controller 140 determines whether to associate or disassociate patient support apparatus 20 with one or more user devices 100, badges 142, and/or locator units 60, it will be understood that the association and/or disassociation process may alternatively, partially, and/or additionally, be carried out by patient support apparatus server 84. In such embodiments, controller 140 forwards relative position information regarding these devices to patient support apparatus server 84 so that server 84 can determine whether to associate a particular device with a particular patient support apparatus 20. In such embodiments, server 84 sends a message back to patient support apparatus 20 informing it of what devices it should be associated with, and what devices it should not be associated with.

Although FIG. 5 illustrates two user devices 100a and 100b, it will be understood that this is merely for illustration purposes. A single user device (100a or 100b), or more than two user devices (100a or 100b) may be associated with patient support apparatus 20.

In some embodiments, controller 140 is adapted to automatically associate itself with any one or more of the devices disclosed in commonly assigned U.S. patent application 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosure of which is incorporated herein by reference.

In some embodiments, locator units 60 may include additional information stored therein that is shared with patient support apparatus 20 when patient support apparatus 20 becomes associated with the locator unit 60. Such additional information may include location information identifying the relative position of the locator unit 60 with respect to one or more other locator units 60 that are positioned nearby. Additionally or alternatively, the locator units 60 may include information regarding the thickness and/or materials of the wall 62 to which it is attached, wherein such information provides an indication to the patient support apparatus 20 of the amount of attenuation that UWB signals will likely experience when traveling through that wall. Additionally or alternatively, the locator units 60 may include information identifying their general location within the healthcare facility (e.g. room 400, bay A of room 302, hallway X, maintenance area Y, radiology department, emergency department, etc.) and/or information identifying a more specific location of the locator units 60 within the healthcare facility (e.g. a set of X,Y,Z coordinates in a frame of reference that includes all, or a portion of, the healthcare facility; a height on the wall 62, a distance from one or more landmarks and/or architectural features within the healthcare facility, and/or other more specific information). In some embodiments, patient support apparatus 20 is adapted to utilize this information to determine its location within the healthcare facility and/or to determine whether it is positioned on the same side of the wall 62 as a particular locator unit 60. In some embodiments, patient support apparatus 20 and/or locator units 60 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

It will be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a support surface adapted to support a patient;
an ultra-wideband (UWB) transceiver adapted to wirelessly communicate with a user device;
a network transceiver adapted to communicate with a healthcare facility computer network; and
a controller adapted to use UWB communication between the UWB transceiver and the user device to determine if the user device is positioned within a first distance of the patient support apparatus, wherein the controller is adapted to forward data from the user device to a server on the healthcare facility network if the user device is positioned within the first distance of the patient support apparatus, and to not forward the data from the user device to the server if the user device is positioned outside of the first distance of the patient support apparatus.

2. The patient support apparatus of claim 1 wherein the controller is further adapted to use UWB communication between the UWB transceiver and a fixed locator unit to determine if the fixed locator unit is positioned within a second distance of the patient support apparatus.

3. The patient support apparatus of claim 2 wherein the controller is further adapted to forward the data from the user device to the server on the healthcare facility network only if the user device is positioned within the first distance of the patient support apparatus and the patient support apparatus is positioned within the second distance of the fixed locator unit, and to not forward the data from the user device to the server if the user device is positioned outside of the first distance of the patient support apparatus or if the patient support apparatus is positioned outside of the second distance of the fixed locator unit.

4. The patient support apparatus of claim 3 wherein the data includes audio signals generated from a microphone of the user device.

5. The patient support apparatus of claim 1 wherein the user device is one of a cell phone, a tablet computer, or a wireless bed pendant, and the network transceiver is a WiFi transceiver.

6. The patient support apparatus of claim 1 wherein the user device is a wireless bed pendant including a control for transmitting a wireless motion command to the patient support apparatus, wherein the controller is adapted to implement the motion command only if the wireless bed pendant transmits the wireless motion command to the patient support apparatus using at least two different wireless communication protocols, and wherein the at least two different wireless communication protocols include Bluetooth and UWB communications.

7. (canceled)

8. The patient support apparatus of claim 2 wherein the controller is further adapted to receive a locator unit ID from the fixed locator unit when the fixed locator unit is positioned within the second distance of the patient support apparatus, and to forward the locator unit ID to the server.

9. The patient support apparatus of claim 2 wherein the controller is adapted to forward the data to the server by forwarding the data to the fixed locator unit.

10. The patient support apparatus of claim 2 wherein the controller is adapted to forward the data to the server by forwarding the data to the server using the network transceiver.

11. (canceled)

12. The patient support apparatus of claim 1 wherein the data include audio signals generated by a microphone of the user device, the patient support apparatus further includes first Bluetooth transceiver adapted to communicate with a second Bluetooth transceiver integrated into the user device, the audio signals are delivered to the patient support apparatus via the first and second Bluetooth transceivers, and the controller is adapted to receive pairing data from the user device via the UWB transceiver and to use the pairing data to automatically pair the first and second Bluetooth transceivers together if the user device is positioned within the first distance of the patient support apparatus.

13-15. (canceled)

16. A patient support apparatus system comprising a patient support apparatus and a server, wherein the patient support apparatus comprises:
(a) a support surface adapted to support a patient;
(b) an ultra-wideband (UWB) transceiver adapted to wirelessly communicate with a user device;
(c) a network transceiver adapted to communicate with the server; and
(d) a controller adapted to use UWB communication between the UWB transceiver and the user device to determine if the user device is positioned within a first distance of the patient support apparatus, wherein the controller is further adapted to forward a location ID and data from the user device to the server if the user device is positioned within the first distance of the patient support apparatus, and to not forward the location ID and the data from the user device to the server if the user device is positioned outside of the first distance of the patient support apparatus;
wherein the server is adapted use the location ID to determine a destination to which to route the data from the user device.

17. The patient support apparatus system of claim 16 wherein the server is further adapted to communicate with a second server to receive a caregiver ID of a caregiver associated with the patient, to use the caregiver ID to determine the destination to which to route the data, and to communicate with a third server to receive a badge ID of a badge associated with the caregiver, and to route the data to the badge.

18. (canceled)

19. The patient support apparatus system of claim 16 wherein the controller is further adapted to use UWB communication between the UWB transceiver and a fixed locator unit to determine if the fixed locator unit is positioned within a second distance of the patient support apparatus.

20. The patient support apparatus system of claim 19 wherein the controller is further adapted to forward the data from the user device to the server only if the user device is positioned within the first distance of the patient support apparatus and the patient support apparatus is positioned within the second distance of the fixed locator unit, and to not forward the data from the user device to the server if the user device is positioned outside of the first distance of the patient support apparatus or if the patient support apparatus is positioned outside of the second distance of the fixed locator unit.

21. The patient support apparatus system of claim 16 wherein the data includes audio signals generated from a microphone of the user device, and the user device is one of a cell phone, a tablet computer, or a wireless bed pendant.

22. (canceled)

23. The patient support apparatus system of claim 16 wherein the user device is a wireless bed pendant including a control for transmitting a wireless motion command to the patient support apparatus, the controller is adapted to implement the motion command only if the wireless bed pendant transmits the wireless motion command to the patient support apparatus using at least two different wireless communication protocols, and the at least two different wireless communication protocols include Bluetooth and UWB communications.

24. (canceled)

25. The patient support apparatus system of claim 19 wherein the controller is further adapted to receive the location ID from the fixed locator unit when the fixed locator unit is positioned within the second distance of the patient support apparatus.

26-27. (canceled)

28. The patient support apparatus system of claim 21 wherein the patient support apparatus further comprises a first Bluetooth transceiver adapted to communicate with a second Bluetooth transceiver integrated into the user device, and wherein the audio signals are delivered to the patient support apparatus via the first and second Bluetooth transceivers.

29. (canceled)

30. The patient support apparatus system of claim 16 wherein the network transceiver is a WiFi transceiver, and the server is adapted to use the location ID to determine multiple destinations to which to route the data from the user device.

31. (canceled)

32. The patient support apparatus system of claim 30 wherein a first one of the multiple destinations is a display device mounted at a fixed location within healthcare facility, and a second one of the multiple destinations is mobile device adapted to be carried by a caregiver assigned to the patient.

33. (canceled)
